(19) United States
(12) Patent Application Publication
(10) Pub. No.: US 2023/0203103 A1
(43) Pub. Date: Jun. 29, 2023

(54) DIAGNOSIS, PREVENTION AND TREATMENT OF CORONAVIRUS INFECTION

(71) Applicant: EMERGEX VACCINES HOLDING LIMITED, Abingdon (GB)

(72) Inventors: Thomas RADEMACHER, Abingdon (GB); Richard PERRINS, Abingdon (GB); Laurens RADEMACHER, Abingdon (GB)

(21) Appl. No.: 18/000,358

(22) PCT Filed: Jun. 2, 2021
(86) PCT No.: PCT/EP2021/064803

§ 371 Date: Nov. 30, 2022

(30) Foreign Application Priority Data
Jun. 2, 2020 (GB) 2008250.9

(51) Int. Cl. C07K 14/005; C07K 14/74; G01N 33/569; C07K 14/725

(52) U.S. Cl. CPC C07K 14/005; C07K 14/70539; G01N 33/56983; C07K 14/7051; C12N 2770/20022; G01N 2333/165; G01N 2800/26; A61K 39/00

(57) ABSTRACT

The invention relates to coronavirus peptides, and the use of such peptides for the diagnosis, treatment and prevention of coronavirus infection.

This application claims priority from GB 2008250.9, filed on 2 Jun. 2020, the contents and elements of which are herein incorporated by reference for all purposes.

### FIELD OF THE INVENTION

The invention relates to coronavirus peptides, and the use of such peptides for the diagnosis, treatment and prevention of coronavirus infection.

### BACKGROUND TO THE INVENTION

Coronaviruses are a group of related viruses that cause diseases in mammals and birds. Symptoms of coronavirus infections vary between species. For instance, coronavirus infection in chickens causes upper respiratory tract disease, whereas coronavirus infections in cows and pigs tend to cause diarrhoea.

In humans, coronaviruses cause respiratory tract infections. The disease caused by infection with some coronaviruses can be mild, such as the common cold. Other coronaviruses cause more serious and potentially fatal disease, such as SARS, MERS, and COVID-19.

Several studies have identified and characterised target sites for the development of vaccines against coronaviruses. For example, He et al. performed experiments to map the antigenic sites of the SARS coronavirus (1). The nucleocapsid protein of SARS has been characterised extensively in order to identify possible vaccine candidates (2), and dominant T helper cell epitopes have been identified in this protein (3). More recently, potential vaccine targets for COVID-19 Coronavirus have been identified based on previous SARS-CoV immunological studies (4). Nevertheless, no vaccine to prevent or treat human coronavirus infections is yet commercially available. Tests for determining current and/or previous coronavirus infections in a human individual are limited. As the control of outbreaks of coronavirus infection relies on strategies involving accurate testing and/or effective vaccination, the provision of vaccines and tests is highly desirable.

### SUMMARY OF THE INVENTION

The present invention relates to coronavirus-derived peptides that may be used to diagnose, prevent or treat coronavirus infections in humans. The present inventors have identified number of peptides that are conserved between different coronaviruses and are presented by MHC molecules on cells infected with those viruses. Inclusion of one or more such peptides in a vaccine composition may confer protective capability against one or more coronaviruses, and/or the ability to treat existing coronavirus infection. Each of the peptides may also be used to diagnose the presence or absence of coronavirus infection, for instance by detecting in a sample the presence or absence of a molecule (such as a T cell receptor or antibody) that is capable of binding to the peptide. The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2.

Accordingly, the present invention provides a peptide comprising any one of SEQ ID NOs: 1 to 34 or a variant thereof.

The present invention also provides:
a complex comprising a peptide of the invention bound to a MHC molecule;
use of a peptide or complex of the invention in a method of determining the presence or absence of current or previous coronavirus infection in an individual;
use of a peptide or complex of the invention in a method of identifying coronavirus-specific T cells;
use of a peptide or complex of the invention in a method of identifying a coronavirus-specific T cell receptor;
a T cell comprising a T cell receptor that is capable of binding to a peptide comprising any one of SEQ ID NOs: 1 to 34 or a variant thereof;
a vaccine composition comprising a peptide of the invention, or a peptide that is capable of binding to a T cell receptor that is capable of binding to a peptide comprising any one of SEQ ID NOs: 1 to 34 or a variant thereof;
a vaccine composition comprising a polynucleotide encoding a peptide of the invention, or a peptide that is capable of binding to a T cell receptor that is capable of binding to a peptide comprising any one of SEQ ID NOs: 1 to 34 or a variant thereof;
a method of preventing or treating a coronavirus infection, comprising administering the vaccine composition of the invention to an individual infected with, or at risk of being infected with, a coronavirus; and
a vaccine composition of the invention for use in a method of preventing or treating a coronavirus infection in an individual.

### DESCRIPTION OF THE FIGURES

FIG. 1: Absorbance spectra for Example 1.

FIG. 2: DLS data for Example 1.

FIG. 3: A—HPLC method for Example 1. B—results of HPLC for Example 1. EM009-064-01 GNP without any peptides is 3.4%. EM009-064-02: GNP without any peptides is 0%. EM009-064-03: GNP without any peptides is 2.2%.

FIG. 4: LC-MS peptide quantitation for Example 1.

FIG. 5: schematic diagram showing alignment of peptides used in Example 1.

### DETAILED DESCRIPTION OF THE INVENTION

### Peptides

The invention provides a peptide comprising any one of SEQ ID NOs: 1 to 34 or a variant thereof. Variants are defined in detail below. SEQ ID NOs: 1 to 34 are set out in Table 1.

TABLE 1

| SEQ | | | | Protein |
|---|---|---|---|---|
| ID NO: | Sequence | HLA affinity | Viral origin | source |
| 1 | LLNKHIDAYK | A3, A11, A31 | SARS Cov / | N |
| | | | SARS Cov 2 | |
| 2 | APSASAFFGM | B7, B35 | SARS Cov / | N |
| | | | SARS Cov 2 | |
| 3 | AQFAPSASA | B72, A2, A203/A2 | SARS Cov / | N |
| | | | SARS Cov 2 | |
| 4 | AQFAPSASAF | B72, B62, B75 | SARS Cov / | N |
| | | | SARS Cov 2 | |
| 5 | ASAFFGMSR | A68, A11, A31 | SARS Cov / | N |
| | | | SARS Cov 2 | |
| 6 | EVTPSGTWL | A68 | SARS Cov / | N |
| | | | SARS Cov 2 | |
| 7 | FAPSASAFF | B35 | SARS Cov / | N |
| | | | SARS Cov 2 | |
| 8 | GMSRIGMEV | A203/A2, A2 | SARS Cov / | N |
| | | | SARS Cov 2 | |
| 9 | HLRIAGHHL | A30 | SARS Cov 2 | M |
| 10 | KHWPQIAQF | A2403/A2, A23 | SARS Cov / | N |
| | | | SARS Cov 2 | |
| 11 | KTFPPTEPK | A11, A30, A3, A68, A31 | SARS Cov / | N |
| | | | SARS Cov 2 | |
| 12 | KTFPPTEPKK | A11, A30, A3, A68 | SARS Cov / | N |
| | | | SARS Cov 2 | |
| 13 | LITGRLQSL | A203/A2, A2 | SARS Cov / | S |
| | | | SARS Cov 2 | |
| 14 | LLLDRLNQL | A2, A203/A2 | SARS Cov / | N |
| | | | SARS Cov 2 | |
| 15 | LLNKHIDAY | B72, B62, B75 | SARS Cov / | N |
| | | | SARS Cov 2 | |
| 16 | MEVTPSGTW | B44 | SARS Cov / | N |
| | | | SARS Cov 2 | |
| 17 | MEVTPSGTWL | B60, B48, B44 | SARS Cov / | N |
| | | | SARS Cov 2 | |
| 18 | QFAPSASAF | A2403/A24 | SARS Cov / | N |
| | | | SARS Cov 2 | |
| 19 | QFAPSASAFF | A24 | SARS Cov / | N |
| | | | SARS Cov 2 | |
| 20 | QIAQFAPSA | A69 | SARS Cov / | N |
| | | | SARS Cov 2 | |
| 21 | RLNEVAKNL | A2, A203/A2 | SARS Cov / | S |
| | | | SARS Cov 2 | |
| 22 | SAFFGMSRI | A68, B63, A203/A2 | SARS Cov / | N |
| | | | SARS Cov 2 | |
| 23 | SASAFFGMSR | A68, A11, A31 | SARS Cov / | N |
| | | | SARS Cov 2 | |
| 24 | SMWALIISV | A2, A203/A2, A69, A32 | SARS Cov 2 | pp1ab |
| 25 | TKAYNVTQAF | B72 | SARS Cov 2 | N |
| 26 | TLACFVLAAV | A2, A203/A2, A68 | SARS Cov / | M |
| | | | SARS Cov 2 | |
| 27 | TPSGTWLTY | B35, B53, A29 | SARS Cov / | N |
| | | | SARS Cov 2 | |
| 28 | VTPSGTWLTY | A29 | SARS Cov / | N |
| | | | SARS Cov 2 | |
| 29 | GETALALLL | B37, B60, B61, B44, | SARS Cov 2 | N |
| | | B48 | | |
| 30 | QFKDNVILL | Cw6, Cw1 | SARS Cov / | N |
| | | | SARS Cov 2 | |
| 31 | SMWALVISV | A2, A203/A2, A32, A69 | SARS Cov / | Orf lab |
| | | | SARS Cov 2 | |
| 32 | TKQYNVTQAF | B72 | SARS Cov / | N |
| | | | SARS Cov 2 | |
| 33 | GDAALALLL | tbc | SARS Cov / | N |
| | | | SARS Cov 2 | |
| 34 | HLRMAGHSL | A30, B7, B8, B62, B72 | SARS Cov / | M |
| | | | SARS Cov 2 | |

SARS Cov = SARS coronavirus. SARS Cov 2 = SARS coronavirus 2.

To identify SEQ ID NOs: 1 to 34, long peptide sequences which have been shown to cause memory T cell responses in SARS-Cov were used to generate a series of short overlapping peptides 9 to 10 residues long. These sequences where then processed using MHCFlurry to predict their binding affinity to various HLA alleles. Sequences with affinities less than or equal to 100 nM and 100% identity to proteins in SARS-COV2 (NCBI accession number NC_045512) were selected.

The peptide of the invention is thus capable of binding to a MHC class I molecule. The peptide may be derived from the immunoproteosome processing of the viral proteome inside an infected cell. The peptide may be a peptide that is expressed on the surface of one or more coronaviruses, or intracellularly within one or more coronaviruses. The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2. The peptide may be a structural peptide or a functional peptide, such as a peptide involved in the metabolism or replication of the coronavirus. Preferably, the peptide is an internal peptide. Preferably, the peptide is conserved between two or more different coronaviruses or coronavirus serotypes. A peptide is conserved between two or more different coronaviruses or coronavirus serotypes if each of the two or more different coronaviruses or coronavirus serotypes encodes a sequence that is 50% or more (such as 60%, 70%, 75%, 80%, 90%, 95%, 98% or 99%) homologous to the peptide.

The peptide may contain any number of amino acids, i.e. be of any length. Typically, the peptide is about 8 to about 30, 35 or 40 amino acids in length, such as about 9 to about 29, about 10 to about 28, about 11 to about 27, about 12 to about 26, about 13 to about 25, about 13 to about 24, about 14 to about 23, about 15 to about 22, about 16 to about 21, about 17 to about 20, or about 18 to about 29 amino acids in length. The peptide preferably has a length of 9 or 10 amino acids. The peptide may be a polypeptide. The peptide may consist of, or consist essentially of, the amino acid sequence of one of SEQ ID NOs: 1 to 34. The peptide may be chemically derived from a polypeptide coronavirus antigen, for example by proteolytic cleavage. More typically, the coronavirus peptide may be synthesised using methods well known in the art.

The peptide may comprise only one of SEQ ID NOs: 1 to 34 or a variant thereof. Alternatively, the peptide may comprise two or more, such as three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more of SEQ ID NOs: 1 to 34 or a variant thereof, in any combination. The peptide may comprise all of SEQ ID NOs: 1 to 34 or a variant thereof. The peptide may, for example, comprise one or more of SEQ ID NOs 21, 1, 19, 28, 2, 27, 16 and 14. The peptide may, for example, comprise all of SEQ ID NOs 21, 1, 19, 28, 2, 27, 16 and 14.

In certain embodiments, the peptide may comprise any one or more of SEQ ID NOs: 9, 24, and 25.

HLRIAGHHL (SEQ ID NO: 9) is based on the sequence HLRMAGHSL (SEQ ID NO: 34) in SARS-CoV-1 M.

SMWALIISV (SEQ ID NO: 24) is based on the SMWALVISV (SEQ ID NO: 31) sequence in SARS-CoV-1 pplab.

TKAYNVTQAF (SEQ ID NO: 25) is based on the TKQYNVTQAF (SEQ ID NO: 32) sequence in SARS-CoV-1 N.

Without wishing to be bound by any particular theory, the present inventors believe that the substitutions advantageously provide for improved and specific detection of SARS-CoV-2.

The peptide may comprise multiple copies, such as two or more, such as three or more, four or more, five or more, six or more, seven or more, eight or more, nine or more, ten or more copies of one or more of SEQ ID NOs: 1 to 34. Peptides that comprise multiple copies of one or more of SEQ ID NOs: 1 to 34, or that comprise one or more of SEQ ID NOs 1 to 34, may have a length of from about 18 to about 250 amino acids, such as from about 20, about 30, about 40 or about 50 amino acids to about 200, about 150 or about 100 amino acids. In such peptides, the two or more of SEQ ID NOs: 1 to 34, or the multiple copies of one or more of SEQ ID NOs: 1 to 34 may be joined directly to one another, or may be joined by one or more, such as from 2 to about 20 amino acids or about 3 to about 10 amino acids. In the peptide, the linking amino acids typically do not comprise the exact amino acid sequence that links the sequences of two or more of SEQ ID NOs: 1 to 34 in nature.

As well as any one of SEQ ID NOs: 1 to 34 or a variant thereof, the peptide may comprise one or more CD8+ T cell epitopes, one or more CD4+ T cell epitopes and/or one or more B cell epitopes. For example, the peptide may comprise two or more, such as three or more, four or more, five or more, ten or more, fifteen or more, or twenty or more CD8+ T cell epitopes. The peptide may comprise two or more, such as three or more, four or more, five or more, ten or more, fifteen or more, or twenty or more CD4+ T cell epitopes. The peptide may comprise two or more, such as three or more, four or more, five or more, ten or more, fifteen or more, or twenty or more B cell epitopes.

The CD8+ T cell epitope is preferably a CD8+ T cell epitope that does not comprise any one of SEQ ID NOs: 1 to 34 or a variant thereof. The CD8+ T cell epitope may, for example, be a coronavirus CD8+ epitope, i.e. a peptide that is expressed by one or more coronaviruses and that is that is capable of (i) presentation by a class I MHC molecule and (ii) recognition by a T cell receptor (TCR) present on a CD8+ T cell.

Alternatively, the CD8+ T cell epitope may be a CD8+ T cell epitope that is not expressed by one or more coronaviruses.

The CD4+ T cell epitope may, for example, be a coronavirus CD4+ epitope, i.e. a peptide that is expressed by one or more coronaviruses and that is that is capable of (i) presentation by a class II MHC molecule and (ii) recognition by a T cell receptor (TCR) present on a CD4+ T cell. Alternatively, the CD4+ T cell epitope may be an CD4+ T cell epitope that is not expressed by one or more coronaviruses.

The B cell epitope may, for example, be a coronavirus B cell epitope, i.e. a peptide that is expressed by one or more coronaviruses and that is that is capable of recognition by a B cell receptor (BCR) present on a B cell. Alternatively, the B cell epitope may be an B cell epitope that is not expressed by one or more coronaviruses.

The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The one or more coronaviruses may, for example, comprise SARS coronavirus and/or SARS coronavirus 2.

The term “peptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immuno1.159, 3230-3237. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2 may be modified to —NH(Me) or —N(Me)₂).

The term “peptide” also includes peptide variants that increase or decrease the half-life of the peptide in vivo. Examples of analogues capable of increasing the half-life of peptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

### Variants

As set out above, the peptide may comprise a variant of any one of SEQ ID NOs: 1 to 34. The peptide may, for example, comprise a variant of 2 or more, such as 3 or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more of SEQ ID NOs: 1 to 34. The peptide may, for example, comprise a variant of all of SEQ ID NOs: 1 to 34. The peptide may, for example, comprise a variant of one or more of SEQ ID NOs 21, 1, 19, 28, 2, 27, 16 and 14. The peptide may, for example, comprise variant of all of SEQ ID NOs 21, 1, 19, 28, 2, 27, 16 and 14.

A variant of any one of SEQ ID NOs: 1 to 34 may be a sequence that differs from any one of SEQ ID NOs: 1 to 34 by no more than five (such as no more than four, no more than three, no more than two, or no more than one) amino acid(s). Each of the no more than five amino acid differences may be an amino acid substitution, deletion or insertion relative to the relevant sequence selected from SEQ ID NOs: 1 to 34. The amino acid substitution may, for example, be a conservative amino acid substitution.

Preferably, a variant of any one of SEQ ID NOs: 1 to 34 may be a sequence that differs from any one of SEQ ID NOs: 1 to 34 by no more than one amino acid. For example, a variant of a sequence selected from SEQ ID NOs: 1 to 34 may comprise one amino acid substitution, deletion or insertion relative to the relevant sequence. The amino acid substitution may, for example, be a conservative amino acid substitution.

Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 2 below. Where amino acids have similar polarity, this can also be determined by reference to the hydropathy scale for amino acid side chains in Table 3.

TABLE 2
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 3
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

### Complexes

The invention provides complex comprising a peptide of the invention bound to a MHC molecule. The complex therefore comprises a peptide comprising or consisting of any one of SEQ ID NOs: 1 to 34, or a variant thereof, bound to a MHC molecule.

Peptide:MHC binding is well-known in the art. Preferably, the binding between the peptide(s) and MHC molecule(s) comprised in the complex is non-covalent. The binding may be mediated by, for example, electrostatic interaction, hydrogen bonds, van der Waals forces and/or hydrophobic interactions.

The WIC molecule may be a MHC class 1 molecule or a MHC class II molecule. Preferably, the MHC molecule is a WIC class I molecule. The MHC class I molecule may be of any HLA supertype. For example, the WIC class I molecule may be of supertype A2, A203/A2, A23, A24, A2403/A2, A2403/A24, A39, A3, A11, A30, A31, A32, A68, A69, B7, B8, B35, B37, B44, B48, B53, B60, B61, B62, B63, B72, B75, Cw1, or Cw6.

The complex may comprise two or more peptides of the invention, and two or more MHC molecules. For example, the complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention. The complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more WIC molecules. The complex may, for example, comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules respectively. The complex may comprise the same number of peptides of the invention as WIC molecules. The complex may comprise a different number of peptides of the invention from the number WIC molecules. The complex may, for example, comprise four WIC molecules. The complex may comprise or consist of a WIC tetramer. The complex may, for example, comprise twelve WIC molecules. The complex may comprise or consist of a WIC dodecamer.

When the complex comprises two or more peptides of the invention, each of the two or more peptides may be the same. Alternatively, each of the two or more peptides may be different. When the complex comprises three or more peptides of the invention, each of the three or more peptides may be the same. When the complex comprises three or more peptides of the invention, each of the three or more peptides may be different. When the complex comprises three or more peptides of the invention, some of the three or more peptides may be the same and some of the three of more peptides may be different. The complex may, for example, comprise two or more of SEQ ID NOs 21, 1, 19, 28, 2, 27, 16 and 14. The complex may, for example, comprise all of SEQ ID NOs 21, 1, 19, 28, 2, 27, 16 and 14.

When the complex comprises two or more MHC molecules, each of the two or more MHC molecules may be the same. Alternatively, each of the two or more MHC molecules may be different. When the complex comprises three or more peptides of the invention, each of the three or more MHC molecules may be the same. When the complex comprises three or more peptides of the invention, each of the three or more MHC molecules may be different. When the complex comprises three or more MHC molecules, some of the three or more MHC molecules may be the same and some of the three of more MHC molecules may be different. When the complex comprises two or more peptides of the invention and two or more MHC molecules, each peptide may be bound to one of the two or more MHC molecules. That is, each peptide comprised in the complex may be bound to an MHC molecule comprised in the complex. Preferably, each peptide comprised in the complex is bound to a different MHC molecule comprised in the complex. That is, each MHC molecule comprised in the complex is preferably bound to no more than one peptide comprised in the complex. The complex may, however, comprise one or more peptides of the invention that are not bound to an MHC molecule. The complex may comprise one or more MHC molecules that are not bound to a peptide of the invention.

The MHC molecule or molecules comprised in the complex may be linked to one another. For example each of the one or more MHC molecules in the complex may be attached to a backbone molecule or a nanoparticle. The MHC molecule or molecules comprised in the complex may be attached to a dextran backbone. That is, the complex may comprise or consist of an MHC dextramer. Mechanisms for attaching a MHC molecule or molecules to a dextran backbone are known in the art. Any number of MHC molecules may be attached to the dextran backbone. For example, one or more, two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MEW molecules may be attached to the dextran backbone.

The complex may comprise a fluorophore. Fluorophores are well-known in the art and include FITC (fluorescein isothiocyanate), PE (phycoerythrin) and APC (allophycocyanin). The complex may comprise any number of fluorophores. For example, the complex may comprise two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more fluorophores. When the complex comprise multiple fluorophores, the fluorophores comprised in the complex may be the same or different. When the complex comprises a backbone, such as a dextran backbone, the fluorophore is preferably attached to the dextran backbone. Mechanisms for attaching a fluorophore to a dextran backbone are known in the art.

### Uses

The peptide or complex of the invention may be utilised in a number of ways, such as in the uses described below.

### Determining the Presence or Absence of Current or Previous Coronavirus Infection

The invention provides use of a peptide or complex of the invention in a method of determining the presence or absence of current or previous coronavirus infection in an individual.

The method may comprise contacting the peptide or complex with a sample obtained from the individual and determining the presence or absence of binding between the peptide or complex and a molecule comprised in the sample.

The sample may, for example, be a blood sample, a serum sample, a plasma sample, a urine sample, a saliva sample, or a sample obtained by swabbing a mucosal surface present in the individual. Preferably, the sample is a blood sample, a serum sample, or a plasma sample.

The molecule may be a molecule that has an immune function. For example, the molecule may be comprised in the innate immune system or adaptive immune system. Preferably, the molecule has a role in adaptive immunity. The molecule may, for example, be an antibody or antibody fragment. The antibody or antibody fragment may be on the surface of, or comprised in, a B cell. The antibody or antibody fragment may be free in the sample. The molecule may, for example, be a T cell receptor. The T cell receptor may be a CD4+ T cell receptor. The T cell receptor may be a CD8+ T cell receptor. The T cell receptor may be on the surface of, or comprised in, a T cell. The T cell may be a CD4+ T cell. The T cell may be a CD8+ T cell.

Preferably, the binding between the peptide or complex and the molecule is non-covalent. The binding may be mediated by, for example, electrostatic interaction, hydrogen bonds, van der Waals forces and/or hydrophobic interactions. Methods for detecting binding between a peptide or peptide-containing complex and a molecule are well-known in the art in include, for example, enzyme-linked immunosorbent assay (ELISA), enzyme-linked immune absorbent spot (ELISpot), and flow cytometry.

The presence of binding may indicate the presence of current or previous coronavirus infection. The absence of binding may indicate the absence of current or previous coronavirus infection.

In a current coronavirus infection, coronavirus particles or components thereof (e.g. peptides, proteins) may be present within the individual. In a current coronavirus infection, antibodies, B cells, CD8+ T cells and/or CD4+ T cells specific for coronavirus particles or components thereof (e.g. peptides, proteins) may be present within the individual. Preferably, in a current coronavirus infection (i) coronavirus particles or components thereof (e.g. peptides, proteins) and (ii) antibodies, B cells, CD8+ T cells and/or CD4+ T cells specific for coronavirus particles or components (e.g. proteins) thereof are present within the individual.

In a previous coronavirus infection, coronavirus particles or thereof components (e.g. peptides, proteins) may be absent from the individual. In a previous coronavirus infection, antibodies, B cells, CD8+ T cells and/or CD4+ T cells specific for coronavirus particles or components thereof (e.g. peptides, proteins) may be present within the individual. Preferably, in a previous coronavirus infection, coronavirus particles or components thereof (e.g. peptides, proteins) are absent from the individual, and antibodies, B cells, CD8+ T cells and/or CD4+ T cells specific for coronavirus particles or components thereof (e.g. peptides, proteins) are present within the individual.

The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2.

### Identifying Coronavirus-Specific T Cells

The invention provides use of a peptide or complex of the invention in a method of identifying coronavirus-specific T cells. The method may comprise contacting the peptide or complex with a sample obtained from an individual and determining the presence or absence of binding between the peptide or complex and a T cell receptor comprised in the sample.

The sample may, for example, be a blood sample, a serum sample, a plasma sample, a urine sample, a saliva sample, or a sample obtained by swabbing a mucosal surface present in the individual. Preferably, the sample is a blood sample.

The T cell receptor may be a CD4+ T cell receptor. The T cell receptor may be a CD8+ T cell receptor. Preferably, the T cell receptor is a CD8+ T cell receptor.

Preferably, the T cell receptor may be on the surface of, or comprised in, a T cell. The T cell may be a CD4+ T cell. The T cell may be a CD8+ T cell. Preferably, the T cell is a CD8+ T cell.

Preferably, the binding between the peptide or complex and the T cell receptor is non-covalent. The binding may be mediated by, for example, electrostatic interaction, hydrogen bonds, van der Waals forces and/or hydrophobic interactions. Methods for detecting binding between a peptide or peptide-containing complex and a T cell receptor are well-known in the art in include, for example, enzyme-linked immunosorbent assay (ELISA), enzyme-linked immune absorbent spot (ELISpot), and flow cytometry.

The presence of binding may indicate the presence of one or more coronavirus-specific T cells. The absence of binding may indicate the absence of coronavirus-specific T cells.

The individual may be currently infected with the coronavirus. In a current coronavirus infection, coronavirus particles or components thereof (e.g. peptides, proteins) may be present within the individual. In a current coronavirus infection, antibodies, B cells, CD8+ T cells and/or CD4+ T cells specific for coronavirus particles or components thereof (e.g. peptides, proteins) may be present within the individual. Preferably, in a current coronavirus infection (i) coronavirus particles or components thereof (e.g. peptides, proteins) and (ii) antibodies, B cells, CD8+ T cells and/or CD4+ T cells specific for coronavirus particles or components thereof (e.g. peptides, proteins) are present within the individual.

The individual may have been previously infected with the coronavirus, but may not be currently infected with the coronavirus. In a previous coronavirus infection, coronavirus particles or components thereof (e.g. peptides, proteins) may be absent from the individual. In a previous coronavirus infection, antibodies, B cells, CD8+ T cells and/or CD4+ T cells specific for coronavirus particles or components thereof (e.g. peptides, proteins) may be present within the individual. Preferably, in a previous coronavirus infection, coronavirus particles or components thereof (e.g. peptides, proteins) are absent from the individual, and antibodies, B cells, CD8+ T cells and/or CD4+ T cells specific for coronavirus particles or components thereof (e.g. peptides, proteins) are present within the individual. Thus, coronavirus particles or components thereof (e.g. peptides, proteins) may be absent from the individual but antibodies, B cells, CD8+ T cells and/or CD4+ T cells specific for coronavirus particles or components thereof (e.g. peptides, proteins) may be present within the individual

The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2.

### Identifying a Coronavirus-Specific T Cell Receptor

The invention provides use of a peptide or complex of the invention in a method of identifying a coronavirus-specific T cell receptor. The method may comprise contacting the peptide or complex with a T cell receptor and determining the presence or absence of binding between the peptide or complex and the T cell receptor.

The T cell receptor may be a CD4+ T cell receptor. The T cell receptor may be a CD8+ T cell receptor. Preferably, the T cell receptor is a CD8+ T cell receptor.

The T cell receptor may be on the surface of, or comprised in, a T cell. The T cell may be a CD4+ T cell. The T cell may be a CD8+ T cell. Preferably, the T is a CD8+ T cell.

Preferably, the binding between the peptide or complex and the T cell receptor is non-covalent. The binding may be mediated by, for example, electrostatic interaction, hydrogen bonds, van der Waals forces and/or hydrophobic interactions. Methods for detecting binding between a peptide or peptide-containing complex and a T cell receptor are well-known in the art in include, for example, enzyme-linked immunosorbent assay (ELISA), enzyme-linked immune absorbent spot (ELISpot), and flow cytometry.

The presence of binding may indicate that the T cell receptor contacted with the peptide or complex is a coronavirus-specific T cell receptor. The absence of binding may indicate that the T cell receptor contacted with the peptide or complex is not coronavirus-specific T cell receptor.

The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2.

### T Cells

The invention provides a T cell comprising a T cell receptor that is capable of binding to a peptide comprising any one of SEQ ID NOs: 1 to 34 or a variant thereof. Methods for detecting binding between a peptide and a T cell receptor are well-known in the art in include, for example, enzyme-linked immunosorbent assay (ELISA), enzyme-linked immune absorbent spot (ELISpot), and flow cytometry. The T cell receptor may have been identified using the method of identifying a coronavirus-specific T cell receptor described above.

The T cell may be an isolated T cell.

The T cell may be a CD4+ T cell. The T cell receptor may be a CD4+ T cell receptor.

The T cell may be a CD8+ T cell. The T cell receptor may be a CD8+ T cell receptor. Preferably, the T cell is a CD8+ T cell. Preferably the T cell receptor is a CD8+ T cell receptor.

The T cell may be a chimeric antigen receptor (CAR) expressing cell. The T cell receptor may be a CAR.

### Vaccine Compositions

The invention provides a vaccine composition comprising a peptide of the invention, or a peptide that is capable of binding to a T cell receptor that is capable of binding to a peptide comprising any one of SEQ ID NOs: 1 to 34 or a variant thereof. Variants are defined above. The vaccine composition has a number of benefits which will become apparent from the discussion below. The key benefits are though summarised here.

Firstly, the vaccine composition is capable of stimulating an immune response against a coronavirus. Preferably, the immune response is a cellular immune response (e.g. a CD8+ T cell response). CD8+ cytotoxic T lymphocytes (CTLs) mediate viral clearance via their cytotoxic activity against infected cells. Stimulating cellular immunity may therefore provide a beneficial defence against coronavirus infection.

Secondly, the peptides identified by the present inventors are conserved between different coronaviruses (such as SARS coronavirus and SARS coronavirus 2) and may be presented by MHC molecules on cells infected with one or more of those viruses. Inclusion of such conserved peptides in the vaccine composition may confer protective capability against (i) related types of virus, (ii) multiple species of coronavirus and/or (iii) multiple lineages or serotypes of a particular species, i.e. confer cross-protection. 100% homology between viruses is not required for cross-protection to be conferred. Rather, cross-protection may arise following immunisation with a sequence that is, for example, about 50% or more (such as 60%, 70%, 75%, 80%, 90%, 95%, 98% or 99%) homologous to a CD8+ T cell epitope expressed in a cell infected with a different virus, if certain residues are retained in the correct position. A vaccine composition comprising one or more peptides comprising any one of SEQ ID NOs: 1 to 34 or a variant thereof, or a peptide that is capable of binding to a T cell receptor that is capable of binding to a peptide comprising any one of SEQ ID NOs: 1 to 34 or a variant thereof, or a corresponding polynucleotide, may therefore be capable of providing cross-protection against a variety of existing coronaviruses over and above those recited in Table 1. Inclusion of one or more conserved peptides in the vaccine composition may also confer protective capability against emerging coronavirus strains associated with evolution of the coronavirus genome. In this way, a single coronavirus vaccine composition can be used to confer protection against a variety of different coronaviruses. This provides a cost-effective means of controlling the spread of coronavirus infection.

Thirdly, different peptides identified by the present inventors are capable of binding to different HLA supertypes. Inclusion of multiple peptides each capable of binding to a different HLA supertype (or corresponding polynucleotides) results in a vaccine composition that is effective in individuals having different HLA types. In this way, a single coronavirus vaccine composition can be used to confer protection in a large proportion of the human population. This again provides a cost-effective means of controlling the spread of coronavirus infection.

Fourthly, the coronavirus peptide comprised in the vaccine composition of the invention may be attached to a nanoparticle, for example a gold nanoparticle. As described in more detail below, attachment to a nanoparticle reduces or eliminates the need to include an adjuvant in the vaccine composition. Attachment to a nanoparticle also reduces or eliminates the need to include a virus in the vaccine composition Thus, the vaccine composition of the invention is less likely to cause adverse clinical effects upon administration to an individual.

The vaccine composition may comprise two or more peptides according to claim 1, each comprising a different sequence selected from SEQ ID NOs: 1 to 34 or a variant thereof. Each of the peptides may have any of the properties set out in the “Peptides” section above. For instance, each peptide may comprise multiple sequences selected from SEQ ID NOs: 1 to 34 or a variant thereof and, optionally, one or more CD8+ T cell epitopes, one or more CD4+ T cell epitopes and/or one or more B cell epitopes. In one aspect, the vaccine composition may comprise three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more peptides each comprising a different sequence selected from SEQ ID NOs: 1 to 34 or a variant thereof. The vaccine composition may comprise any combination of peptides. The vaccine composition may, for example, comprise 34 peptides each comprising a different sequence selected from SEQ ID NOs: 1 to 34 or a variant thereof.

The vaccine composition may comprise two or more peptides that each are capable of binding to a different T cell receptor that is capable of binding to a peptide comprising any one of SEQ ID NOs: 1 to 34 or a variant thereof, wherein each different T cell receptor is capable of binding to a different sequence selected from SEQ ID NOs: 1 to 34 or a variant thereof. Each of the peptides may have any of the properties set out in the “Peptides” section above. For instance, each peptide comprised in the vaccine composition may be capable of binding to multiple different T cell receptors that are each capable of binding to a peptide comprising any one of SEQ ID NOs: 1 to 34 or a variant thereof. The vaccine may comprise one or more CD8+ T cell epitopes, one or more CD4+ T cell epitopes and/or one or more B cell epitopes. In one aspect, the vaccine composition may comprise three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more peptides that each are capable of binding to a different T cell receptor that is capable of binding to a peptide comprising any one of SEQ ID NOs: 1 to 34 or a variant thereof. The vaccine composition may comprise any combination of peptides. The vaccine composition may, for example, comprise 34 peptides that are each capable of binding to a different T cell receptor that is capable of binding to a peptide comprising any one of SEQ ID NOs: 1 to 34 or a variant thereof.

### Cross-Protection

SEQ ID NOs: 1 to 34 identified by the present inventors are expressed by multiple coronaviruses. The vaccine composition may therefore elicit a protective immune response against more than one coronavirus, such as SARS coronavirus and SARS coronavirus 2. In other words, the vaccine composition of the invention may elicit an immune response that is cross-protective against a number of different coronaviruses. Each of the different coronaviruses may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus.

An immune response generated by vaccination with a composition that comprises an epitope that is 100% homologous with a sequence from another virus may protect against subsequent infection with that virus. An immune response generated by vaccination with a composition that comprises an epitope that is about 50% or more (such as 60%, 70%, 75%, 80%, 90%, 95%, 98% or 99%) homologous with a sequence encoded by another virus may protect against subsequent infection with that virus. In some cases, the protective effect is associated with the conservation of certain residues between the epitope and the sequence encoded by the other virus. Immunisation with a vaccine composition of the invention may therefore induce a protective immune response against a variety of viruses not mentioned in Table 1, such as other coronaviruses.

Accordingly, the vaccine composition of the invention may have built-in cross-species and/or cross-genus efficacy, i.e. be a cross-protective vaccine composition. Thus, a single coronavirus vaccine composition of the invention may be used to confer protection against a variety of different coronaviruses. This provides a cost-effective means of controlling the spread of coronavirus infection.

Inclusion of conserved peptides in the vaccine composition may confer protective capability against emerging coronavirus strains associated with evolution of the coronavirus genome. This may assist in the long-term control of coronavirus infection.

Interaction with HLA Supertypes

The vaccine composition may comprise at least two peptides which each interact with a different HLA supertype. Including a plurality of such peptides in the vaccine composition allows the vaccine composition to elicit an immune response (such as a CD8+ T cell response) in a greater proportion of individuals to which the vaccine composition is administered. This is because the vaccine composition should be capable of eliciting an immune response in all individuals of an HLA supertype that interacts with one of the peptides comprised in the vaccine composition. Each peptide may interact with A2, A203/A2, A23, A24, A2403/A2, A2403/A24, A39, A3, All, A30, A31, A32, A68, A69, B7, B8, B35, B37, B44, B48, B53, B60, B61, B62, B63, B72, B75, Cw1, or Cw6, or any other HLA supertype know in the art. Any combination of peptides is possible.

The vaccine composition may comprise at least one peptide which interacts at least two different HLA supertypes. Again, this allows the vaccine composition to elicit an immune response (such as a CD8+ T cell response) in a greater proportion of individuals to which the vaccine composition is administered. The vaccine composition may comprise at least two, at least three, at least four, at least five, at least ten, at least fifteen, at least twenty, at least 25 or at least 30 peptides that each interact with at least two different HLA subtypes. Each peptide may interact, for example, with at least two, at least three, at least four, at least five, at least six, at least 7, at least 8, at least 9, at least 10, at least 11, at least 12, at least 13, at least 14, or at least 15 different HLA supertypes. Each peptide may interact with two or more from A2, A2, A203/A2, A23, A24, A2403/A2, A2403/A24, A39, A3, A11, A30, A31, A32, A68, A69, B7, B8, B35, B37, B44, B48, B53, B60, B61, B62, B63, B72, B75, Cw1, and Cw6, or any other HLA supertype known in the art, in any combination.

Preferably, the vaccine composition comprises a peptide that interacts with A3, A11 and A31. In this case, the vaccine composition may, for example, comprise a peptide that comprises SEQ ID NO: 1 and/or 11. The vaccine composition may, for example, comprise a peptide that comprises SEQ ID NO: 1 and a peptide that comprises SEQ ID NO: 11.

Preferably, the vaccine composition comprises a peptide that interacts with B7 and B35. In this case, the vaccine composition may, for example, comprise a peptide that comprises SEQ ID NO: 2.

Preferably, the vaccine composition comprises a peptide that interacts with B72, A2 and A203/A2. In this case, the vaccine composition may, for example, comprise a peptide that comprises SEQ ID NO: 3.

Preferably, the vaccine composition comprises a peptide that interacts with B72, B62 and B75. In this case, the vaccine composition may, for example, comprise a peptide that comprises SEQ ID NO: 4 and/or 15. The vaccine composition may, for example, comprise a peptide that comprises SEQ ID NO: 4 and a peptide that comprises SEQ ID NO: 15.

Preferably, the vaccine composition comprises a peptide that interacts with A68, A11 and A31. In this case, the vaccine composition may, for example, comprise a peptide that comprises SEQ ID NO: 5 and/or 11. The vaccine composition may, for example, comprise a peptide that comprises SEQ ID NO: 5 and a peptide that comprises SEQ ID NO: 11.

Preferably, the vaccine composition comprises a peptide that interacts with A203/A2 and A2. In this case, the vaccine composition may, for example, comprise a peptide that comprises SEQ ID NO: 3, 8, 13, 14, 21, 24 and/or 26. The vaccine composition may, for example, comprise a peptide that comprises SEQ ID NO: 3, a peptide that comprises SEQ ID NO: 8, a peptide that comprises SEQ ID NO: 13, a peptide that comprises SEQ ID NO: 14, a peptide that comprises SEQ ID NO: 21, a peptide that comprises SEQ ID NO: 24 and a peptide that comprises SEQ ID NO: 26.

Preferably, the vaccine composition comprises a peptide that interacts with A2403/A2 and A23. In this case, the vaccine composition may, for example, comprise a peptide that comprises SEQ ID NO: 10.

Preferably, the vaccine composition comprises a peptide that interacts with A11, A30, A3, A68 and A31. In this case, the vaccine composition may, for example, comprise a peptide that comprises SEQ ID NO: 11.

Preferably, the vaccine composition comprises a peptide that interacts with A11, A30, A3 and A68. In this case, the vaccine composition may, for example, comprise a peptide that comprises SEQ ID NO: 12.

Preferably, the vaccine composition comprises a peptide that interacts with B60, B48 and B44. In this case, the vaccine composition may, for example, comprise a peptide that comprises SEQ ID NO: 17.

Preferably, the vaccine composition comprises a peptide that interacts with A68, B63 and A203/A2. In this case, the vaccine composition may, for example, comprise a peptide that comprises SEQ ID NO: 22.

Preferably, the vaccine composition comprises a peptide that interacts with A2, A203/A2, A69 and A32. In this case, the vaccine composition may, for example, comprise a peptide that comprises SEQ ID NO: 24 or SEQ ID NO: 31.

Preferably, the vaccine composition comprises a peptide that interacts with A2, A203/A2 and A68. In this case, the vaccine composition may, for example, comprise a peptide that comprises SEQ ID NO: 26.

Preferably, the vaccine composition comprises a peptide that interacts with B35, B53, A29. In this case, the vaccine composition may, for example, comprise a peptide that comprises SEQ ID NO: 27.

Preferably, the vaccine composition comprises a peptide that interacts with B37, B60, B61, B44 and B48. In this case, the vaccine composition may, for example, comprise a peptide that comprises SEQ ID NO: 29.

Preferably, the vaccine composition comprises a peptide that interacts with Cw6 and Cw1. In this case, the vaccine composition may, for example, comprise a peptide that comprises SEQ ID NO: 30.

Preferably, the vaccine composition comprises a peptide that interacts with A30, B7, B8, B62 and B72. In this case, the vaccine composition may, for example, comprise a peptide that comprises SEQ ID NO: 34.

### Nanoparticles

The peptide, or one or more peptides, may be attached to a nanoparticle, for example in the vaccine composition of the invention. Any other peptides further comprised in the vaccine composition may also be attached to a nanoparticle. Attachment to a nanoparticle, for example a gold nanoparticle, is beneficial.

As set out above, attachment of the peptide to a nanoparticle (such as a gold nanoparticle) reduces or eliminates the need to include a virus or an adjuvant in the vaccine composition. The nanoparticles may contain immune “danger signals” that help to effectively induce an immune response to the peptides. The nanoparticles may induce dendritic cell (DC) activation and maturation, required for a robust immune response. The nanoparticles may contain non-self components that improve uptake of the nanoparticles and thus the peptides by cells, such as antigen presenting cells. Attachment of a peptide to a nanoparticle may therefore enhance the ability of antigen presenting cells to stimulate virus-specific T and/or B cells. Attachment to a nanoparticle also facilitates delivery of the vaccine compositions via the subcutaneous, intradermal, transdermal and oral/buccal routes, providing flexibility in administration.

Nanoparticles are particles between 1 and 100 nanometers (nm) in size which can be used as a substrate for immobilising ligands. In the vaccine compositions of the invention, the nanoparticle may have a mean diameter or mean core diameter of 1 to 100, 20 to 90, 30 to 80, 40 to 70 or 50 to 60 nm. Preferably, the nanoparticle has a mean diameter or mean core diameter of 5 to 40 nm, such as 10 to 30 nm, or 20 to 32 nm. Preferably, the nanoparticle has a mean diameter or mean core diameter of 5 nm. A mean diameter or mean core diameter of 5 to 40 nm facilitates uptake of the nanoparticle to the cytosol. The mean diameter or mean core diameter can be measured using techniques well known in the art such as transmission electron microscopy.

Nanoparticles suitable for the delivery of antigen, such as a peptide of the invention, are known in the art. Methods for the production of such nanoparticles are also known.

The nanoparticle may, for example, be a polymeric nanoparticle, an inorganic nanoparticle, a liposome, an immune stimulating complex (ISCOM), a virus-like particle (VLP), or a self-assembling protein. The nanoparticle is preferably a calcium phosphate nanoparticle, a silicon nanoparticle or a gold nanoparticle.

The nanoparticle may be a polymeric nanoparticle. The polymeric nanoparticle may comprise one or more synthetic polymers, such as poly(d,l-lactide-co-glycolide) (PLG), poly(d,l-lactic-coglycolic acid) (PLGA), poly(g-glutamic acid) (g-PGA)m poly(ethylene glycol) (PEG), or polystyrene. The polymeric nanoparticle may comprise one or more natural polymers such as a polysaccharide, for example pullulan, alginate, inulin, and chitosan. The use of a polymeric nanoparticle may be advantageous due to the properties of the polymers that may be include in the nanoparticle. For instance, the natural and synthetic polymers recited above may have good biocompatibility and biodegradability, a non-toxic nature and/or the ability to be manipulated into desired shapes and sizes. The polymeric nanoparticle may form a hydrogel nanoparticle. Hydrogel nanoparticles are a type of nano-sized hydrophilic three-dimensional polymer network. Hydrogel nanoparticles have favourable properties including flexible mesh size, large surface area for multivalent conjugation, high water content, and high loading capacity for antigens. Polymers such as Poly(L-lactic acid) (PLA), PLGA, PEG, and polysaccharides are particularly suitable for forming hydrogel nanoparticles.

The nanoparticle may be an inorganic nanoparticle. Typically, inorganic nanoparticles have a rigid structure and are non-biodegradable. However, the inorganic nanoparticle may be biodegradable. The inorganic nanoparticle may comprise a shell in which an antigen may be encapsulated. The inorganic nanoparticle may comprise a core to which an antigen may be covalently attached. The core may comprise a metal. For example, the core may comprise gold (Au), silver (Ag) or copper (Cu) atoms. The core may be formed of more than one type of atom. For instance, the core may comprise an alloy, such as an alloy of Au/Ag, Au/Cu, Au/Ag/Cu, Au/Pt, Au/Pd or Au/Ag/Cu/Pd. The core may comprise calcium phosphate (CaPO₄). The core may comprise a semiconductor material, for example cadmium selenide.

Other exemplary inorganic nanoparticles include carbon nanoparticles and silica-based nanoparticles. Carbon nanoparticles have good biocompatibility and can be synthesized into nanotubes and mesoporous spheres. Silica-based nanoparticles (SiNPs) are biocompatible and can be prepared with tuneable structural parameters to suit their therapeutic application.

The nanoparticle may be a silicon nanoparticle, such as an elemental silicon nanoparticle. The nanoparticle may be mesoporous or have a honeycomb pore structure. Preferably, the nanoparticle is an elemental silicon particle having a honeycomb pore structure. Such nanoparticles are known in the art and offer tuneable and controlled drug loading, targeting and release that can be tailored to almost any load, route of administration, target or release profile. For example, such nanoparticles may increase the bioavailability of their load, and/or improve the intestinal permeability and absorption of orally administered actives. The nanoparticles may have an exceptionally high loading capacity due to their porous structure and large surface area. The nanoparticles may release their load over days, weeks or months, depending on their physical properties. Since silicon is a naturally occurring element of the human body, the nanoparticles may elicit no response from the immune system. This is advantageous to the in vivo safety of the nanoparticles.

Any of the SiNPs described above may be biodegradable or non-biodegradable. A biodegradable SiNP may dissolve to orthosilic acid, the bioavailable form of silicon. Orthosilic acid has been shown to be beneficial for the health of bones, connective tissue, hair, and skin.

The nanoparticle may be a liposome. Liposomes are typically formed from biodegradable, non-toxic phospholipids and comprise a self-assembling phospholipid bilayer shell with an aqueous core. A liposome may be an unilameller vesicle comprising a single phospholipid bilayer, or a multilameller vesicle that comprises several concentric phospholipid shells separated by layers of water. As a consequence, liposomes can be tailored to incorporate either hydrophilic molecules into the aqueous core or hydrophobic molecules within the phospholipid bilayers. Liposomes may encapsulate antigen within the core for delivery. Liposomes may incorporate viral envelope glycoproteins to the shell to form virosomes. A number of liposome-based products are established in the art and are approved for human use.

The nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine) and saponin (such as Quil A from the tree Quillaia saponaria). ISCOMs have traditionally been used to entrap viral envelope proteins, such as envelope proteins from herpes simplex virus type 1, hepatitis B, or influenza virus.

The nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

The nanoparticle may be a self-assembling protein. For instance, the nanoparticle may comprise ferritin. Ferritin is a protein that can self-assemble into nearly-spherical 10 nm structures. The nanoparticle may comprise major vault protein (MVP). Ninety-six units of MVP can self-assemble into a barrel-shaped vault nanoparticle, with a size of approximately 40 nm wide and 70 nm long.

The nanoparticle may be a calcium phosphate (CaPO₄) nanoparticle. CaPO₄ nanoparticles may comprise a core comprising one or more (such as two or more, 10 or more, 20 or more, 50 or more, 100 or more, 200 or more, or 500 or more) molecules of CaPO₄. CaPO₄ nanoparticles and methods for their production are known in the art. For instance, a stable nano-suspension of CAP nanoparticles may be generated by mixing inorganic salt solutions of calcium and phosphates in pre-determined ratios under constant mixing.

The CaPO₄ nanoparticle may have an average particle size of about 80 to about 100 nm, such as about 82 to about 98 nm, about 84 to about 96 nm, about 86 to about 94 nm, or about 88 to about 92 nm. This particle size may produce a better performance in terms of immune cell uptake and immune response than other, larger particle sizes. The particle size may be stable (i.e. show no significant change), for instance when measured over a period of 1 month, 2 months, 3 months, 6 months, 12 months, 18 months, 24 months, 36 months or 48 months.

CaPO₄ nanoparticles can be co-formulated with one or multiple antigens either adsorbed on the surface of the nanoparticle or co-precipitated with CaPO₄ during particle synthesis. For example, a peptide, such as a peptide of the invention, may be attached to the CaPO₄ nanoparticle by dissolving the peptide in DMSO (for example at a concentration of about 10 mg/ml), adding to a suspension of CaPO₄ nanoparticles together with N-acetyl-glucosamine (GlcNAc) (for example at 0.093 mol/L and ultra-pure water, and mixing at room temperature for a period of about 4 hours (for example, 1 hour, 2 hours, 3 hours, 5 hours, 6 hours, 7 hours, 8 hours, 9 hours or 10 hours).

The vaccine composition may comprise about 0.15 to about 0.8%, such as 0.2 to about 0.75%, 0.25 to about 0.7%, 0.3 to about 0.6%, 0.35 to about 0.65%, 0.4 to about 0.6%, or 0.45 to about 0.55%, CaPO₄ nanoparticles. Preferably the vaccine composition comprises about 0.3% CaPO₄ nanoparticles.

CaPO₄ nanoparticles have a high degree of biocompatibility due to their chemical similarity to human hard tissues such as bone and teeth. Advantageously, therefore, CaPO₄ nanoparticles are non-toxic when used for therapeutic applications. CaPO₄ nanoparticles are safe for administration via intramuscular, subcutaneous, oral, or inhalation routes. CaPO₄ nanoparticles are also simple to synthesise commercially. Furthermore, CaPO₄ nanoparticles may be associated with slow release of antigen, which may enhance the induction of an immune response to a peptide attached to the nanoparticle. CaPO₄ nanoparticles may be used both as an adjuvant, and as a drug delivery vehicle.

The nanoparticle may be a gold nanoparticle. Gold nanoparticles are known in the art and are described in particular in WO 2002/32404, WO 2006/037979, WO 2007/122388, WO 2007/015105 and WO 2013/034726. The gold nanoparticle attached to each peptide may be a gold nanoparticle described in any of WO 2002/32404, WO 2006/037979, WO 2007/122388, WO 2007/015105 and WO 2013/034726.

Gold nanoparticles comprise a core comprising a gold (Au) atom. The core may further comprise one or more Fe, Cu or Gd atoms. The core may be formed from a gold alloy, such as Au/Fe, Au/Cu, Au/Gd, Au/Fe/Cu, Au/Fe/Gd or Au/Fe/Cu/Gd. The total number of atoms in the core may be 100 to 500 atoms, such as 150 to 450, 200 to 400 or 250 to 350 atoms. The gold nanoparticle may have a mean diameter of 1 to 100, 20 to 90, 30 to 80, 40 to 70 or 50 to 60 nm. Preferably, the gold nanoparticle has a mean diameter of 20 to 40 nm.

The nanoparticle may comprise a surface coated with alpha-galactose and/or beta-GlcNAc. For instance, the nanoparticle may comprise a surface passivated with alpha-galactose and/or beta-GlcNAc. In this case, the nanoparticle may, for example, be a nanoparticle which comprises a core including metal and/or semiconductor atoms. For instance, the nanoparticle may be a gold nanoparticle. Beta-GlcNAc is a bacterial pathogen-associated-molecular pattern (PAMP), which is capable of activating antigen-presenting cells. In this way, a nanoparticle comprising a surface coated or passivated with Beta-GlcNAc may non-specifically stimulate an immune response. Attachment of the flavivirus peptide comprising one or more of the CD8+ T cell epitopes set out in SEQ ID NOs: 1 to 23 or a variant thereof to such a nanoparticle may therefore improve the immune response elicited by administration of the vaccine composition of the invention to an individual.

One or more ligands other than the peptide may be linked to the nanoparticle, which may be any of the types of nanoparticle described above. The ligands may form a “corona”, a layer or coating which may partially or completely cover the surface of the core. The corona may be considered to be an organic layer that surrounds or partially surrounds the nanoparticle core. The corona may provide or participate in passivating the core of the nanoparticle. Thus, in certain cases the corona may be a sufficiently complete coating layer to stabilise the core. The corona may facilitate solubility, such as water solubility, of the nanoparticles of the present invention.

The nanoparticle may comprise at least 10, at least 20, at least 30, at least 40 or at least 50 ligands. The ligands may include one or more peptides, protein domains, nucleic acid molecules, lipidic groups, carbohydrate groups, anionic groups, or cationic groups, glycolipids and/or glycoproteins. The carbohydrate group may be a polysaccharide, an oligosaccharide or a monosaccharide group (e.g. glucose). One or more of the ligands may be a non-self component, that renders the nanoparticle more likely to be taken up by antigen presenting cells due to its similarity to a pathogenic component. For instance, one or more ligands may comprise a carbohydrate moiety (such as a bacterial carbohydrate moiety), a surfactant moiety and/or a glutathione moiety. Exemplary ligands include thiolated glucose, N-acetylglucosamine (GlcNAc), glutathione, 2′-thioethyl-β-D-glucopyranoside and 2′-thioethyl-D-glucopyranoside. Preferred ligands include glycoconjugates, which form glyconanoparticles

Linkage of the ligands to the core may be facilitated by a linker. The linker may comprise a thiol group, an alkyl group, a glycol group or a peptide group. For instance, the linker may comprise C2-C15 alkyl and/or C2-C15 glycol. The linker may comprise a sulphur-containing group, amino-containing group, phosphate-containing group or oxygen-containing group that is capable of covalent attachment to the core. Alternatively, the ligands may be directly linked to the core, for example via a sulphur-containing group, amino-containing group, phosphate-containing group or oxygen-containing group comprised in the ligand.

### Attachment to Nanoparticles

The peptide may be attached at its N-terminus to the nanoparticle. Typically, the peptide is attached to the core of the nanoparticle, but attachment to the corona or a ligand may also be possible.

The peptide may be directly attached to the nanoparticle, for example by covalent bonding of an atom in a sulphur-containing group, amino-containing group, phosphate-containing group or oxygen-containing group in the peptide to an atom in the nanoparticle or its core.

A linker may be used to link the peptide to the nanoparticle. The linker may comprise a sulphur-containing group, amino-containing group, phosphate-containing group or oxygen-containing group that is capable of covalent attachment to an atom in the core. For example, the linker may comprise a thiol group, an alkyl group, a glycol group or a peptide group.

The linker may comprise a peptide portion and a non-peptide portion. The peptide portion may comprise the sequence X₁X₂Z₁, wherein X₁ is an amino acid selected from A and G; X₂ is an amino acid selected from A and G; and Z₁ is an amino acid selected from Y and F. The peptide portion may comprise the sequence AAY or FLAAY (SEQ ID NO: 44). The peptide portion of the linker may be linked to the N-terminus of the peptide. The non-peptide portion of the linker may comprise a C2-C15 alkyl and/ a C2-C15 glycol, for example a thioethyl group or a thiopropyl group.

The linker may be (i) HS-(CH₂)₂-CONH-AAY; (ii) HS-(CH₂)₂—CONH-LAAY (SEQ ID NO: 43); (iii) HS-(CH2)₃-CONH-AAY; (iv) HS-(CH2)₃—CONH-FLAAY (SEQ ID NO: 44); (v) HS-(CH₂)₁₀—(CH₂OCH₂)₇—CONH-AAY; and (vi) HS-(CH₂)₁₀—(CH₂OCH₂)₇—CONH-FLAAY (SEQ ID NO: 44). In this case, the thiol group of the non-peptide portion of the linker links the linker to the core.

Other suitable linkers for attaching a peptide to a nanoparticle are known in the art, and may be readily identified and implemented by the skilled person.

When the vaccine composition comprises more than one peptide, two or more (such as three or more, four or more, five or more, ten or more, or twenty or more) of the peptides may be attached to the same nanoparticle. Two or more (such as three or more, four or more, five or more, ten or more, or twenty or more) of the peptides may each be attached to different nanoparticle. The nanoparticles to which the peptides are attached may though be the same type of nanoparticle. For instance, each peptide may be attached to a gold nanoparticle. Each peptide may be attached to a CaPO₄ nanoparticle. The nanoparticle to which the peptides are attached may be a different type of nanoparticle. For instance, one peptide may be attached to a gold nanoparticle, and another peptide may be attached to a CaPO₄ nanoparticle.

### Polynucleotide Vaccines

The invention provides a vaccine composition comprising a polynucleotide encoding a peptide according to claim 1, or a peptide that is capable of binding to a T cell receptor that is capable of binding to a peptide comprising any one of SEQ ID NOs: 1 to 34 or a variant thereof.

The vaccine composition may comprise a polynucleotide encoding two or more peptides of the invention, each comprising a different sequence selected from SEQ ID NOs: 1 to 34 or a variant thereof.

The vaccine composition may comprise a polynucleotide encoding two or more peptides that each are capable of binding to a different T cell receptor that is capable of binding to a peptide comprising any one of SEQ ID NOs: 1 to 34 or a variant thereof, wherein each different T cell receptor is capable of binding to a different sequence selected from SEQ ID NOs: 1 to 34 or a variant thereof.

The vaccine composition may comprise two or more polynucleotides each encoding a peptide of the invention, wherein each peptide comprises a different sequence selected from SEQ ID NOs: 1 to 34 or a variant thereof.

The vaccine composition may comprise two or more polynucleotides each encoding a peptide that is capable of binding to a T cell receptor that is capable of binding to a peptide comprising any one of SEQ ID NOs: 1 to 34 or a variant thereof, wherein each peptide is capable of binding to a different T cell receptor that is capable of binding to a peptide comprising any one of SEQ ID NOs: 1 to 34 or a variant thereof and each different T cell receptor is capable of binding to a different sequence selected from SEQ ID NOs: 1 to 34 or a variant thereof.

The polynucleotide may be DNA. The polynucleotide may be RNA. For example, the polynucleotide may be mRNA.

### Medicaments, Methods of Treatment and Therapeutic Use

The invention provides a method of preventing or treating a coronavirus infection, comprising administering a vaccine composition of the invention to an individual infected with, or at risk of being infected with, a coronavirus. The invention also provides a vaccine composition of the invention for use in a method of preventing or treating a coronavirus infection in an individual.

The coronavirus infection may, for example, be a coronavirus infection that is implicated in a human epidemic or pandemic. The coronavirus infection may, for example, be a coronavirus infection of zoonotic origin. The coronavirus infection may, for example, be an infection with a member of the genus Betacoronavirus. The coronavirus may, for example, be an infection with a member of subgenus Sarbecoronavirus. The coronavirus infection may be, for example, a SARS coronavirus infection or a SARS coronavirus 2 infection.

The vaccine composition may be provided as a pharmaceutical composition. The pharmaceutical composition preferably comprises a pharmaceutically acceptable carrier or diluent. The pharmaceutical composition may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA.

The vaccine composition or pharmaceutical composition may be administered by any route. Suitable routes include, but are not limited to, the intravenous, intramuscular, intraperitoneal, subcutaneous, intradermal, transdermal and oral/buccal routes.

Compositions may be prepared together with a physiologically acceptable carrier or diluent. Typically, such compositions are prepared as liquid suspensions of peptides and/or peptide-linked nanoparticles. The peptides and/or peptide-linked nanoparticles may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions may contain minor amounts of auxiliary substances such as wetting or emulsifying agents, and/or pH buffering agents.

The peptides or peptide-linked nanoparticles are administered in a manner compatible with the dosage formulation and in such amount will be therapeutically effective. The quantity to be administered depends on the subject to be treated, the disease to be treated, and the capacity of the subject's immune system. Precise amounts of nanoparticles required to be administered may depend on the judgement of the practitioner and may be peculiar to each subject.

Any suitable number of peptides or peptide-linked nanoparticles may be administered to a subject. For example, at least, or about, 0.2×10⁶, 0.25×10⁶, 0.5×10⁶, 1.5×10⁶, 4.0×10⁶ or 5.0×10⁶ peptides or peptide-linked nanoparticles per kg of patient may administered. For example, at least, or about, 10⁵, 10⁶, 10⁷, 10⁸, 10⁹ peptides or peptide-linked nanoparticles may be administered. As a guide, the number of peptides or peptide-linked nanoparticles to be administered may be from 10⁵ to 10⁹, preferably from 10⁶ to 10⁸.

### EXAMPLE 1

### Introduction

Coronavirus peptides were attached to gold nanoparticles as described below.

8 peptides were selected: P77, P81, P83, P86, P92, P96, P99 and P100, as shown in Table 4.

TABLE 4

| | | SEQ | | | |
|---|---|---|---|---|---|
| | | ID | | | |
| Peptide | Sequence | NO | Protein | HLA type | Corresponds to |
| P77 | AAYRLNEVAKNL | 35 | S | A2, | SEQ ID NO: 21 + |
| | | | | A203/A2 | N-terminal AAY |
| | | | | | (peptide portion |
| | | | | | of linker) |
| P81 | AAYLLNKHIDAYK | 36 | N | A3, A11, | SEQ ID NO: 1 + |
| | | | | A31 | N-terminal AAY |
| | | | | | (peptide portion |
| | | | | | of linker) |
| P83 | AAYQFAPSASAFF | 37 | N | A24 | SEQ ID NO: 19 + |
| | | | | | N-terminal AAY |
| | | | | | (peptide portion |
| | | | | | of linker) |
| P86 | AAYVTPSGTWLTY | 38 | N | A29 | SEQ ID NO: 28 + |
| | | | | | N-terminal AAY |
| | | | | | (peptide portion |
| | | | | | of linker) |
| P92 | AAYAPSASAFFGM | 39 | N | B7, B35 | SEQ ID NO: 2 + |
| | | | | | N-terminal AAY |
| | | | | | (peptide portion |
| | | | | | of linker) |
| P96 | AAYTPSGTWLTY | 40 | N | B35, B53, | SEQ ID NO: 27 + |
| | | | | A29 | N-terminal AAY |
| | | | | | (peptide portion |
| | | | | | of linker) |
| P99 | AAYMEVTPSGTW | 41 | N | B44 | SEQ ID NO: 16 + |
| | | | | | N-terminal AAY |
| | | | | | (peptide portion |
| | | | | | of linker) |
| P100 | AAYLLLDRLNQL | 42 | N | A2, | SEQ ID NO: 14 + |
| | | | | A203/A2 | N-terminal AAY |
| | | | | | (peptide portion |
| | | | | | of linker) |

The aim of this experiment was to make a 100 mg Au scale batch for the toxicology study based on a test GNP EM009-062-01 at 4 mg Au scale. The total peptides loading started with 5eq per NP (estimated as per 100Au atom/NP) for the ligands exchange.

### Methods

| Material | Supplier | Batch/R number | Comments |
|---|---|---|---|
| 8 COIVD-19 peptides | ChinaPeptides | | |
| Base GNP | ChemCon | CC190252A | [Au] = 3.193 g/L |
| Water | Grifols | 8470006470169 | Water for injectons |
| DMSO | Fisher | 165487 | |
| 15 mL Falcon tubes | Thermo Scientific | 339650 Lot: J4AF317116 | |
| 50 mL Falcon tubes | Thermo Scientific | 339653 Lot: J4AF318119 | |
| Nalgene Syringe Filter, Sterile | Thermo Scientific | 723-2520 Lot: 1711052513 | SFCA membrane, 0.2 μm |
| 20 mL Syringe | Fisher Scientific | 300629 Lot: 1908237 | BD Luer-Lok |
| 1.5 mL Microcentrifuge Tubes | Thermo Scientific | 3451 Lot: 19231845 | Low Retention |
| NUNC ™ Serological pipettes | Thermo Scientific | 170367 Lot: M349670 | Sterile, disposable, 10 mL |
| Amicon ® Ultra −15 | Merck Millipore Ltd | UFC901096 Lot: R0BB00196 | 15 mL, 10 kDa |

### Calculations

Table 5 concerns 8 peptides' DMSO solution preparation. Table 5 lists the amounts weighted out and volume of DMSO added to produce a 1 mM stock assuming peptide content/purity of 90%. Peptide quantitation is problematic, while peptide purity is quoted for these peptides no ‘peptide content’ was given these can be as low as 50% sometimes, hence our use of 90% as an estimate followed up by in house HPLC quantitation.

TABLE 5

DMSO

| Peptides | MW | Amount (mg) | Volume (mL) |
|---|---|---|---|
| P77 | 1450 | 7.62 | 4.73 |
| P81 | 1608 | 9.54 | 5.34 |
| P83 | 1466 | 11.05 | 6.78 |
| P86 | 1518 | 8.12 | 4.81 |
| P92 | 1379 | 8.14 | 5.31 |
| P96 | 1418 | 9.63 | 6.11 |
| P99 | 1401 | 12.52 | 8.04 |
| P100 | 1491 | 12.12 | 7.32 |

1 mL of base GNP weighed 1.003 g, so 100 mg Au=1.003*(100/3.193)=31.4 g, base particle was weighted out for accuracy. The actual volumes taken are based on the following calculation: 100 mg Au=505 μmole Au=5.05 μmole NP at a 5 fold excess peptide/NP=25.25 μmole total peptide, but 8 peptides so=3.16 μmole each peptide.

The peptides were weighted and dissolved in a laminar flow hood (LAF), a freshly sealed bottle of DMSO was used for solubilisation. The peptide stocks above were assayed by HPLC. As an example, for P77 we obtained an area of 50.1 instead of expected 70 AUC (as defined by Tyr or Tryp residue absorbance at 278 nm). Instead of being 1 mM the peptide was determined to be 0.716 mM, therefore to get 3.16 μmole P77 we would need 4.43 ml as shown in the table below. Some peptide stocks had disulphide peptides. These were ignored as only thiol peptide areas were quantitated.

The actual volumes of 8 peptides' DMSO solutions taken for ligands exchange are given in Table 6 below.

TABLE 6
EM009-064-01

| | μmol |
|---|---|
| 100 mg Au: | 5.077 |
| 5eq: | 25.385 |
| each peptide: | 3.173 |

DMSO Coupling

| | Area | Theoretical area | Volume (mL) |
|---|---|---|---|
| P77 | 50.1 | 70 | 4.433 |
| P81 | 91.1 | 140 | 4.876 |
| P83 | 74.1 | 70 | 2.998 |
| P86 | 289 | 440 | 4.831 |
| P92 | 67 | 70 | 3.315 |
| P96 | 374 | 440 | 3.733 |
| P99 | 329.5 | 370 | 3.563 |
| P100 | 40.1 | 70 | 5.539 |
| | | Total | 33.288 |

### Procedure

31.4 g of ChemCon base GNP was weighed in a 50 mL sterile falcon tube. All 8 peptides DMSO solutions were added together in a 250 mL glass round-bottomed flask. The ChemCon Tox base GNP was then added and briefly mixed, the vessel was nitrogen flushed and sealed. This ligands exchange solution mixture was kept stirring at 300 rpm, 30° C. in a water bath for 3 h.

After 3 h, the dark brown GNP solution was concentrated on 15 mL 10 kDa Amicon Tubes (×8), then washed with sterile ‘water for injection’ all additions were performed in LAF (×5,4000 G for 8 min per centrifuge DMSO was kept below 15% in Amicon devices). GNP solution was collected from Amicon tubes into 12 1.5 mL Eppendorf tubes, which were then centrifuged at 17 G for 2 min to remove any aggregates. Supernatant from each Eppendorf tube was combined and filtered through two 0.2 μm sterile Nalgene syringe filters (around 5 mL GNP solution per filter). The final sterile GNP solution (EM009-064-01) was 10 mL and it was then kept at 4° C. 200 μL of this final GNP solution was removed and kept separately for the analyses.

It had been noticed that there was some GNP material on the Amicon tube membrane and, after the final hard spin at 17kG for 2 min of the main product, a pellet was found at the bottom of each Eppendorf tube. Later investigations showed that these precipitated GNPs can be re-suspended in 0.2M carbonate buffer (CB pH 10.22). All precipitated GNPs from Amicon and Eppendorf tubes were re-suspended in 0.2M carbonate buffer (pH 10.22), and concentrated on the same 8 Amicon tubes from the previous day, then washed with 0.2M CB one more time, and followed by washing with water (×5, 4000 G for 8 min per centrifuge). Almost no GNP was observed in the Amicon tubes. After centrifuging at 17 kG for 2 min (no precipitation was observed), this GNP solution (EM009-064-02) was 2.7 mL and was kept at 4° C. for further analysis. It can be seen below that the Au yield was 62.6% for the main sterile prep, with 24.9% lost as insoluble aggregates. To solubilise the aggregated material and greatly increase the yield a 0.2M carbonate buffer (pH 10.22) wash pre water washes can be used.
EM009-064-01: supernatant GNP solution from the big toxicology batch.
EM009-064-02: precipitated GNP after treating with 0.2M CB (pH 10.22) from the main toxicology batch.
EM009-064-03: test mixture of EM0090-64-01 (50 μL) and 02 (13.52 μL).

### Analysis

### Gold Assay

Results of a gold assay are shown in Table 7.

TABLE 7

| Batch | [Au] (g/L) | Volume (mL) | Yield (%) |
|---|---|---|---|
| Dutch Tox | 3.193 | — | — |
| Base GNP | | | |
| EM009-064-01 | 6.263 | 10 | 62.6 |
| EM009-064-02 | 9.215 | 2.7 | 24.9 |
| EM009-064-03 | 7.304 | — | — |

### Absorbance Spectra

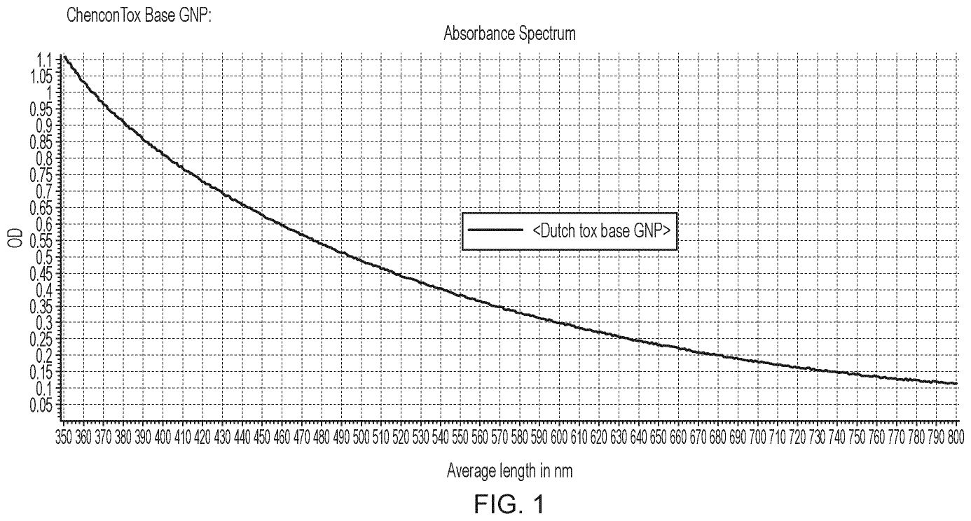

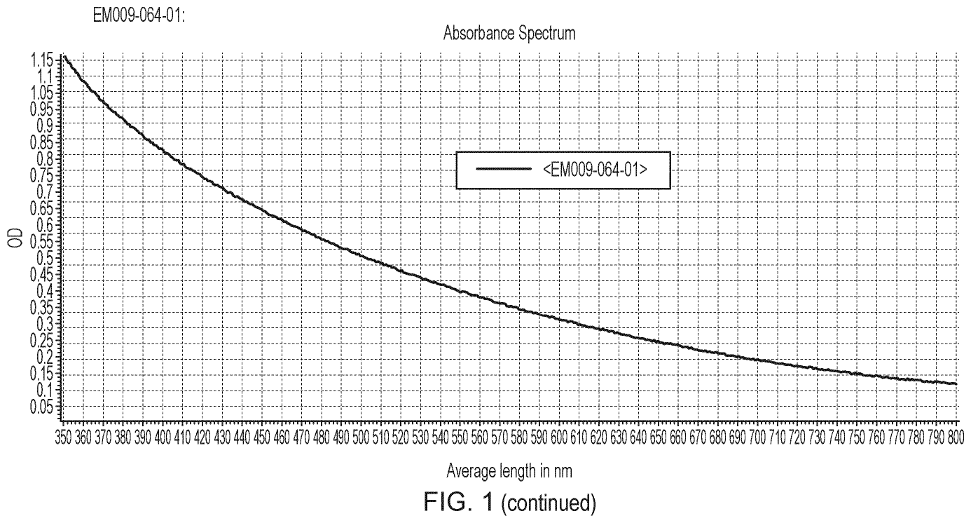

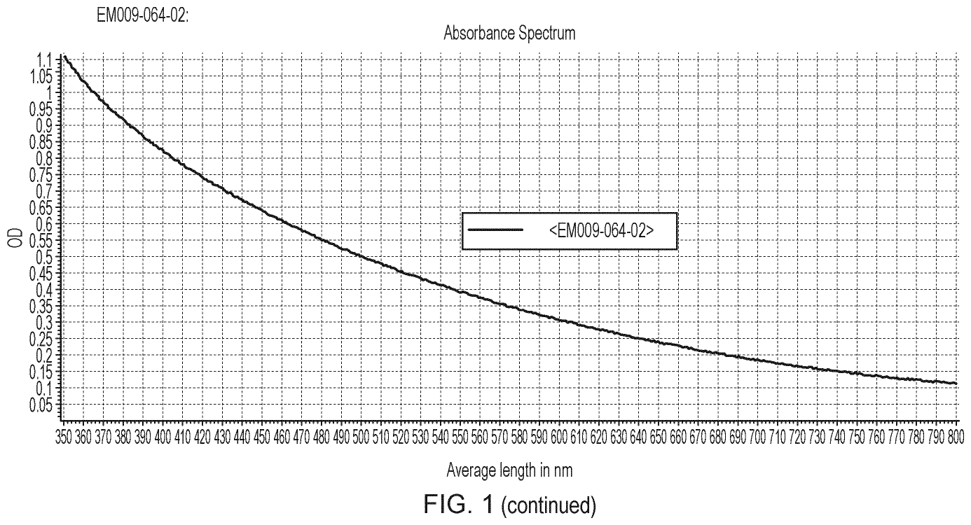

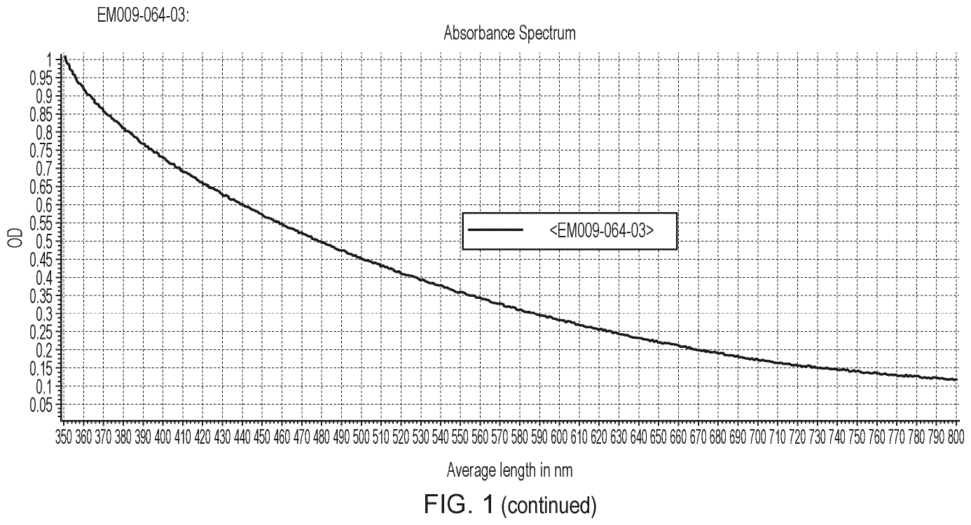

Absorbance spectra for ChenconTox Base GNP, EM009-009-064-01, EM009-064-02 and EM009-064-03 are shown in FIG. 1.

No plasmon band at 520 nm could be seen in ChemCon Tox Base GNP, EM009-064-01, 02 and 03 batches.

### DLS

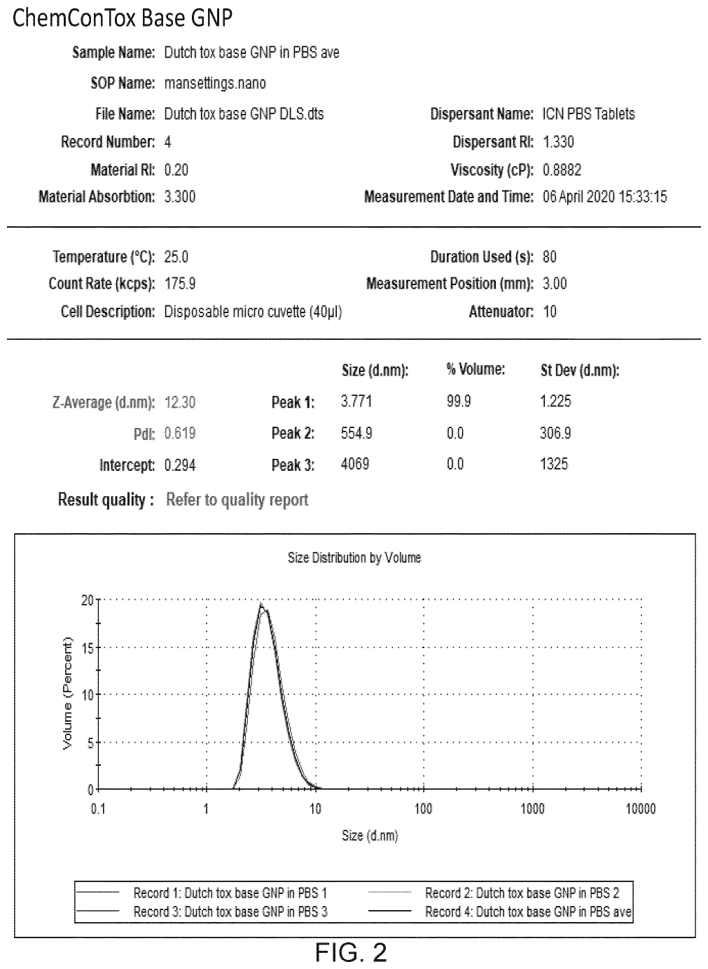

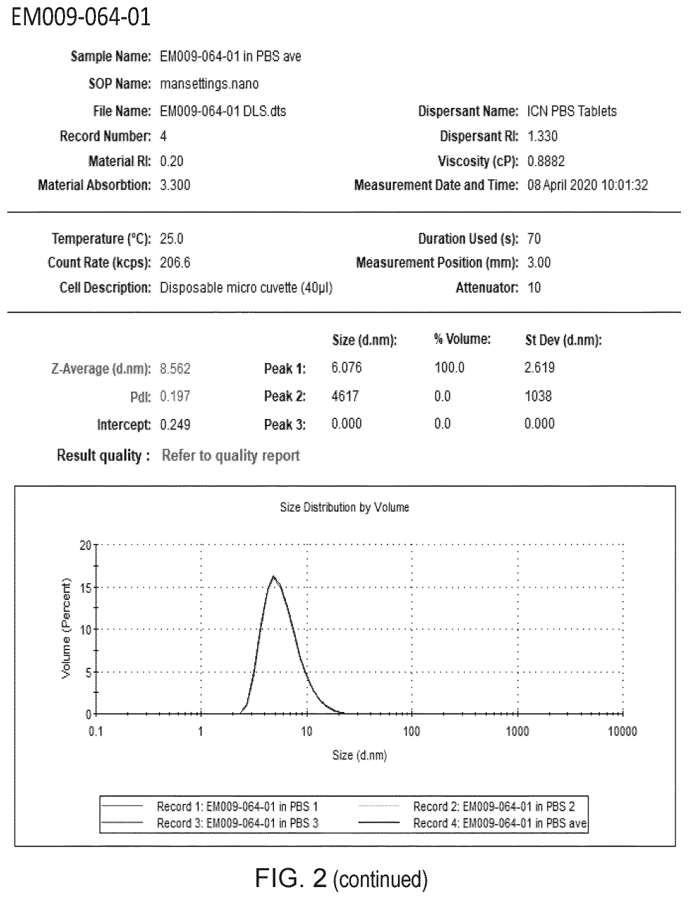

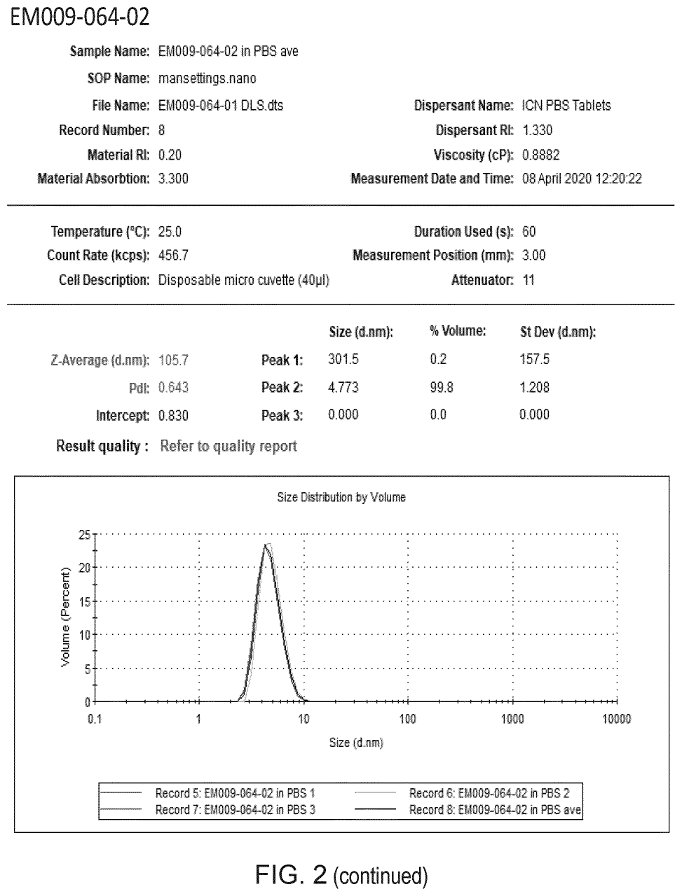

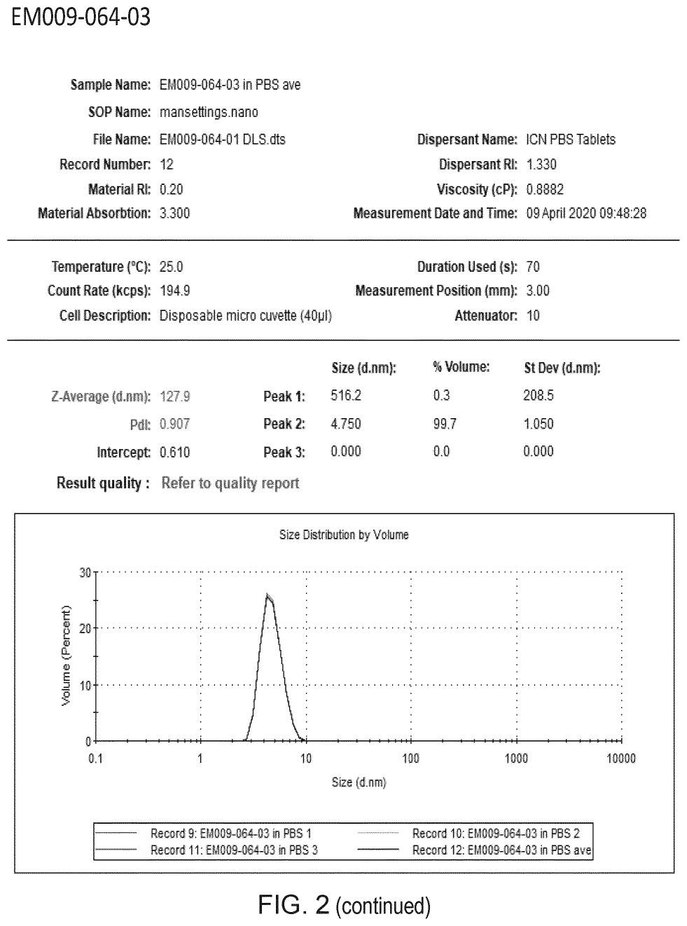

DLS results are shown in FIG. 2, and summarised below.

For ChemConTox Base GNP, size=3.77 nm (n=3), SD=±1.22 nm. For EM009-064-01, size=6.08 nm (n=3), SD=±2.62 nm. For EM009-064-02, size=4.77 nm (n=3), SD=±1.21 nm. For EM009-064-03, size=4.75 nm (n=3), SD=±1.05 nm.

### HPLC of Nanoparticle 400 nm

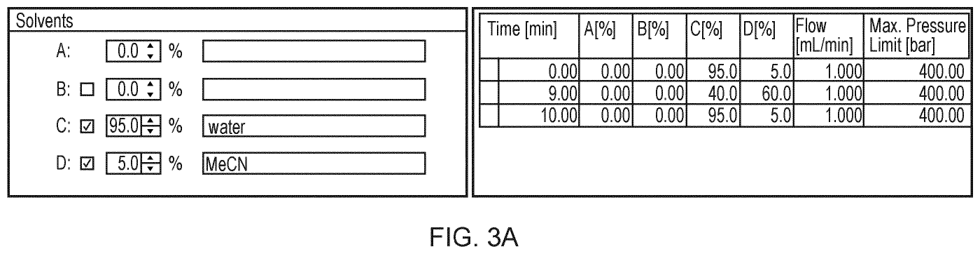

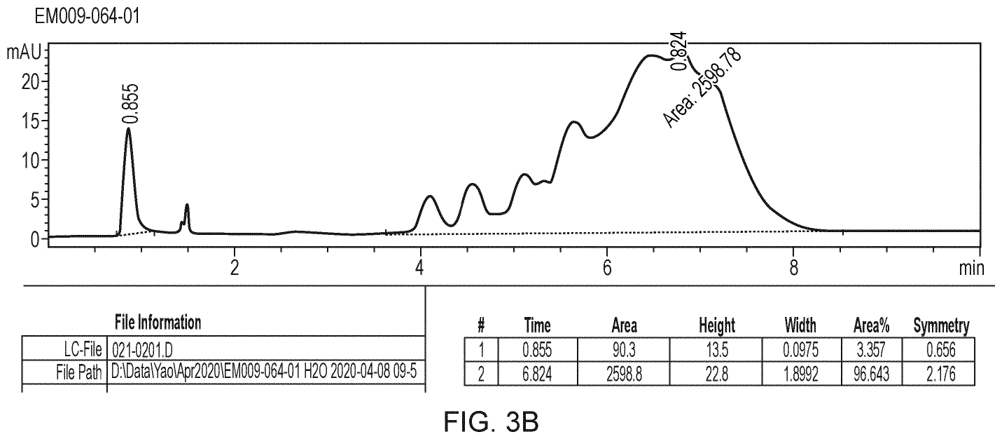

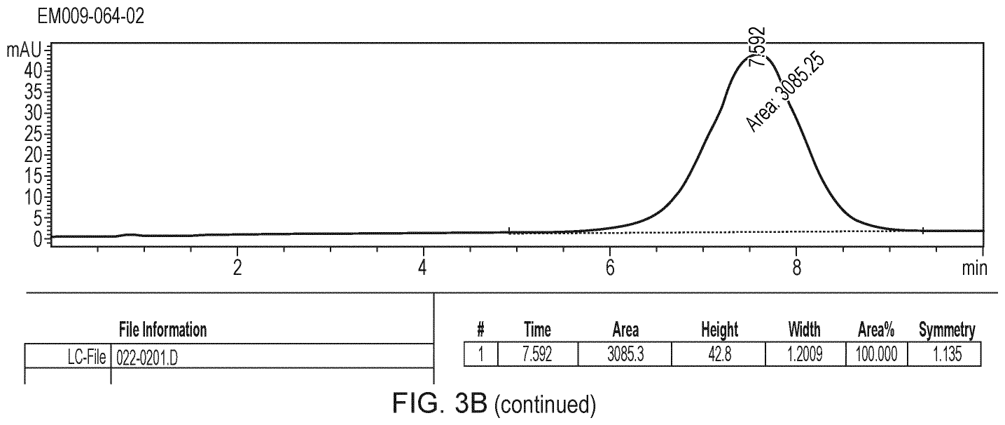

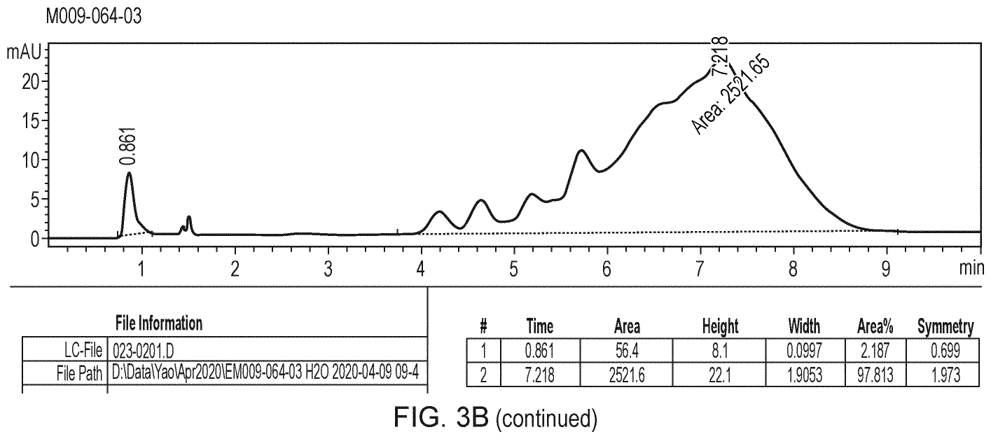

The method is summarised in FIG. 3. Sample preparation is shown in Table 8 below.

TABLE 8

| Batch No. | GNP (μL) | H₂O (μL) |
|---|---|---|
| EM009-064-01 | 2.55 | 37.45 |
| EM009-064-02 | 1.74 | 38.26 |
| EM009-064-03 | 2.19 | 37.81 |

For each batch, 16 μg of Au was in 40 μL of water. On HPLC, 10 μL of this GNP solution was injected to the column, which gives 4 μg Au per injection for each batch.

HPLC results showed all three samples had excellent peptide incorporation (>96%). EM009-064-01 GNP without any peptides is 3.4%. EM009-064-02 GNP without any peptides is 0%. EM009-064-03: GNP without any peptides is 2.2%.

LC MS Peptide Quantitation

Sample preparation is set out in Table 9 below.

TABLE 9

| | LC-Ms (0.25 g/L) inject 32 μL → 8 μg Au | | |
|---|---|---|---|
| | | 0.1M | |
| Batch No. | GNP (μL) | TCEP (μL) | DMSO (μL) |
| EM009-064-01 | 3.99 | 40.00 | 56.01 |
| EM009-064-02 | 2.71 | 40.00 | 57.29 |
| EM009-064-03 | 3.42 | 40.00 | 56.58 |

For each batch, 25 μg of Au was incubated with 0.1M TCEP at 40° C. for 4 h, then it was topped up to 100 μL with DMSO. On LC-MS, 32 μL of this GNP solution was injected to the column, which gives 8 μg Au per injection for each batch.

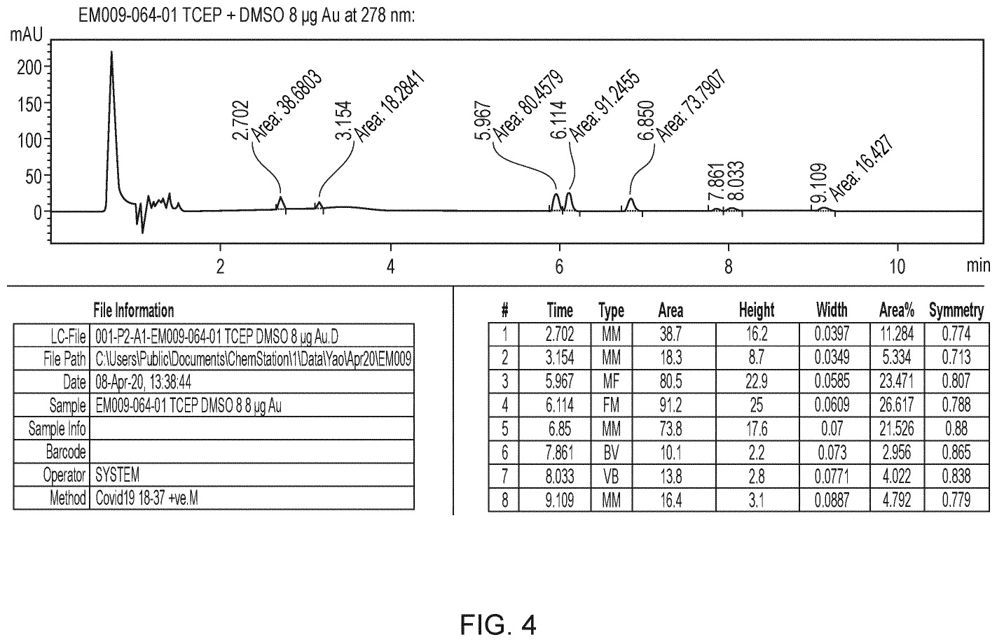

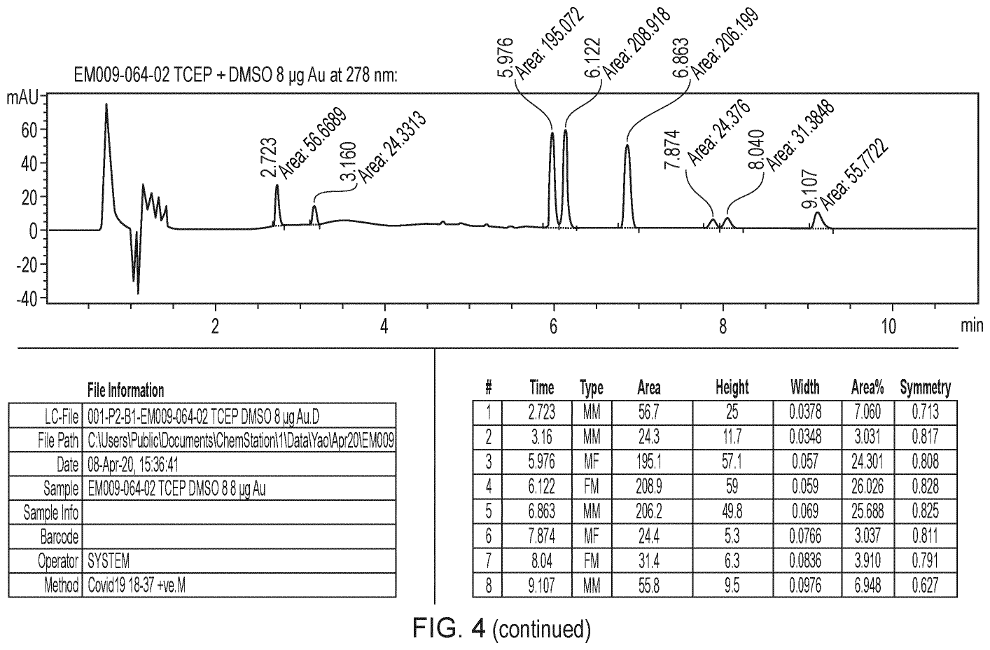

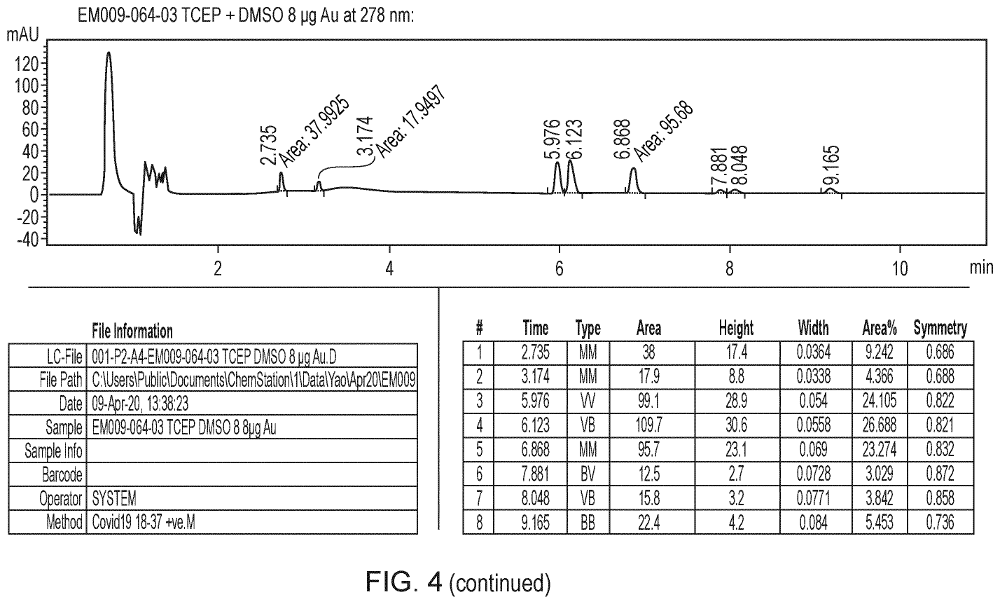

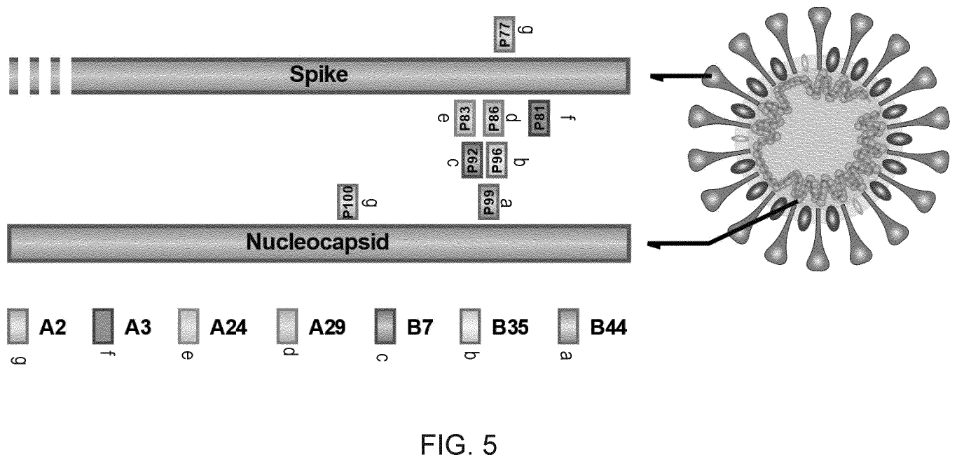

Results are shown in FIG. 4.

Individual peptide loading from all 3 batches is shown in Table 10.

TABLE 10

| | EM009-064-01 | | EM009-064-02 | | EM009-064-03 | |
|---|---|---|---|---|---|---|
| | TCEP DMSO | | TCEP DMSO | | TCEP DMSO | |
| Peptides | Area | nmol | Area | nmol | Area | nmol |
| P81 | 38.7 | 0.28 | 56.7 | 0.41 | 38 | 0.27 |
| P77 | 18.3 | 0.26 | 24.3 | 0.35 | 17.9 | 0.26 |
| P99 | 80.5 | 0.22 | 195.1 | 0.53 | 99.1 | 0.27 |
| P96 | 91.2 | 0.21 | 208.9 | 0.47 | 109.7 | 0.25 |
| P86 | 73.8 | 0.17 | 206.2 | 0.47 | 95.7 | 0.22 |
| P83 | 10.1 | 0.14 | 24.4 | 0.35 | 12.5 | 0.18 |
| P92 | 13.8 | 0.20 | 31.4 | 0.45 | 15.8 | 0.23 |
| P100 | 16.4 | 0.23 | 55.8 | 0.80 | 22.4 | 0.32 |
| Total | nmol | 1.71 | nmol | 3.82 | nmol | 1.99 |
| | Eq/NP | 4.20 | eq | 9.40 | eq | 4.89 |

### Conclusion

The particle sizes of both batches are good. However, EM009-064-01 batch (supernatant GNP solution) has slightly bigger size than EM009-064-02 (precipitated GNP treated with CB). No plasmon band at 520 nm were observed in any of the three batches.

HPLC of whole GNP product showed minimal GNP without any peptides at all <4%. The big single peak from EM009-064-02 HPLC chromatogram indicates this batch has higher loading of hydrophobic peptides (P92 and/or P100). Probably that is why this batch precipitated from water solution during the Amicon wash.

EM009-064-01: the total 8 peptides loading is 4.2 eq (started with 5eq). LC-MS showed P81 and P77 had slightly higher loading, whereas P86 and P83 had slightly lower loading than expected.

EM009-064-02: the total 8 peptides loading is 9.4 eq (started with 5eq). LC-MS showed P77 and P83 had slightly lower loading, whereas P100 had a very high loading, almost doubled when compared with other peptides. P100 is very hydrophobic, such high loading, explains why this batch precipitated from water wash during EM009-064-01 purification. However, after washed with 0.2M CB, this precipitated GNP can be re-suspended in aqueous solution again. This batch was resolubilized in non-sterile conditions and is not intended to be mixed with the main EM009-064-01 product.

EM009-064-03: 50 μL of EM009-064-01 was mixed with 13.52 μL EM009-064-02. The final peptide loading is 4.89. P83 loading is still a bit low and P100 loading is still high, but this mixed batch showed better overall peptide loading results when compared with EM009-064-01 and 02 batches.

EM009-064-01 has about 62.6 mg Au and EM009-064-02 has about 24.9 mg Au. If combining both batches together, it will give an 87.5% gold recovery yield. In future during final GNP purification on Amicon, 0.2M CB should be used to keep GNP in the solution to increase the final gold yield and improve peptide ratios/levels.

In terms of product quantities, 10 ml of material at 6.3 mg/ml Au and a total peptide content of 1.34 μmole/ml, i.e.13.4 μmole total peptide provides material for over 1600 doses of approximately 1 nmole of each peptide.

### REFERENCES

1. He Y, Zhou Y, Wu H, Kou Z, Liu S, Jiang S. Mapping of antigenic sites on the nucleocapsid protein of the severe acute respiratory syndrome coronavirus. J Clin Microbiol. 2004 Nov; 42(11): 5309-14. doi: 10.1128/JCM.42.11.5309-5314.2004.
2. Liu S J, Leng C H, Lien S P, et al. Immunological characterizations of the nucleocapsid protein based SARS vaccine candidates. Vaccine. 2006; 24(16): 3100-3108. doi:10.1016/j.vaccine.2006.01.058.
3. Zhao J, Huang Q, Wang W, Zhang Y, Lv P, Gao X M. Identification and characterization of dominant helper T-cell epitopes in the nucleocapsid protein of severe acute respiratory syndrome coronavirus. J Virol. 2007; 81(11): 6079-6088. doi: 10.1128/JVI.02568-06
4. Ahmed S F, Quadeer A A, McKay M R. Preliminary Identification of Potential Vaccine Targets for the COVID-19 Coronavirus (SARS-CoV-2) Based on SARS-CoV Immunological Studies. Viruses. 2020; 12(3):254. https://doi.org/10.3390/v12030254.

1. A peptide comprising any one of SEQ ID NOs: 9, 1 to 8, 10 to 34 or a variant thereof.

2. A complex comprising a peptide according to claim 1 bound to a MHC molecule.

3. A complex according to claim 2, wherein the complex comprises two or more peptides according to claim 1 and two or more MHC molecules.

4. A complex according to claim 3, wherein each peptide is bound to one of the two or more MHC molecules.

5. A complex according to claim 3 or 4, wherein each of the two or more MHC molecules is attached to a dextran backbone.

6. A complex according to claim 5, wherein the complex further comprise a fluorophore, optionally wherein the fluorophore is attached to the dextran backbone.

7. A complex according to any one of claims 3 to 7, wherein the complex comprises two or more of SEQ ID NOs 21, 1, 19, 28, 2, 27, 16 and 14 or a variant thereof, optionally wherein the complex comprises SEQ ID NOs 21, 1, 19, 28, 2, 27, 16 and 14 or a variant thereof.

8. Use of a peptide according to claim 1 or a complex according to any one of claims 2 to 7 in a method of determining the presence or absence of current or previous coronavirus infection in an individual.

9. A use according to claim 8, wherein the method comprises contacting the peptide or complex with a sample obtained from the individual and determining the presence or absence of binding between the peptide or complex and a molecule comprised in the sample.

10. A use according to claim 9, wherein the molecule is an antibody or a T cell receptor.

11. A use according to claim 9 or 10, wherein the presence of binding indicates the presence of current or previous coronavirus infection, and/or the absence of binding indicates the absence of current or previous coronavirus infection.

12. Use of a peptide according to claim 1 or a complex according to any one of claims 2 to 7 in in a method of identifying coronavirus-specific T cells.

13. A use according to claim 12, wherein the method comprises contacting the peptide or complex with a sample obtained from an individual and determining the presence or absence of binding between the peptide or complex and a T cell receptor comprised in the sample.

14. A use according to claim 13, wherein the individual is currently infected with the coronavirus.

15. A use according to claim 14, wherein the individual was previously, but is not currently, infected with the coronavirus.

16. Use of a peptide of claim 1 or complex of any one of claims 2 to 7 in a method of identifying a coronavirus-specific T cell receptor.

17. A use according to claim 16, wherein the method comprises contacting the peptide or complex with a T cell receptor and determining the presence or absence of binding between the peptide or complex and the T cell receptor.

18. A use according to claim 17, wherein the presence of binding indicates that the T cell receptor is a coronavirus-specific T cell receptor, and/or the absence of binding indicates that the T cell receptor is not coronavirus-specific T cell receptor.

19. A T cell comprising a T cell receptor that is capable of binding to a peptide comprising any one of SEQ ID NOs: 9, 1 to 8, 10 to 34 or a variant thereof.

20. A vaccine composition comprising a peptide according to claim 1, or a peptide that is capable of binding to a T cell receptor as defined in claim 19.

21. A vaccine composition according to claim 20, which comprises:
(a) two or more peptides according to claim 1, each comprising a different sequence selected from SEQ ID NOs: 9, 1 to 8, 10 to 34 or a variant thereof; or
(b) two or more peptides that each are capable of binding to a different T cell receptor as defined in claim 19, wherein each different T cell receptor is capable of binding to a different sequence selected from SEQ ID NOs: 9, 1 to 8, 10 to 34 or a variant thereof.

22. A vaccine composition according to claim 21, wherein the vaccine composition comprises two or more peptides each comprising a different sequence selected from SEQ ID NOs 21, 1, 19, 28, 2, 27, 16 and 14or a variant thereof.

23. A vaccine composition according to claim 21, wherein each of the two or more peptides interacts with a different HLA supertype.

24. A vaccine composition according to any one of claims 20 to 23, which comprises at least one peptide according to claim 20, wherein the peptide interacts with at least two different HLA supertypes.

25. A vaccine composition according to claim 23 or 24, wherein the at least two different HLA supertypes are:
(i) selected from A2, A203/A2, A23, A24, A2403/A2, A2403/A24, A39, A3, A11, A30, A31, A32, A68, A69, B7, B8, B35, B37, B44, B48, B53, B60, B61, B62, B63, B72, B75, Cwl and Cw6;
(ii) A3, A11 and A31;
(iii) B7 and B35;
(iv) B72, A2 and A203/A2;
(v) B72, B62 and B75;
(vi) A68, A11 and A31;
(vii) A203/A2 and A2;
(viii) A2403/A2 and A23;
(ix) A11, A30, A3, A68 and A31;
(x) A11, A30, A3 and A68;
(xi) B60, B48 and B44;
(xii) A68, B63 and A203/A2
(xiii) A2, A203/A2, A69 and A32
(xiv) A2, A203/A2 and A68;
(xv) B35, B53, A29;
(xvi) B37, B60, B61, B44 and B48;
(xvii) Cw6 and Cw1; and
(xviii) A30, B7, B8, B62 and B72.

26. A vaccine composition according to any one of claims 20 to 25, wherein the peptide is attached to a nanoparticle.

27. A vaccine composition according to any one of claims 21 to 26, wherein each of the two or more peptides is attached to a nanoparticle.

28. A vaccine composition according to claim 26 or 27, wherein the nanoparticle is a gold nanoparticle, a calcium phosphate nanoparticle, or a silicon nanoparticle, optionally wherein the gold nanoparticle is coated with alpha-galactose and/or beta-GlcNAc.

29. A vaccine composition according to any one of claims 26 to 28, wherein the peptide is attached to the nanoparticle via a linker.

30. A vaccine composition comprising a polynucleotide encoding a peptide according to claim 1, or a peptide that is capable of binding to a T cell receptor as defined in claim 19.

31. A vaccine composition according to claim 30, which comprises a polynucleotide encoding:
(a) two or more peptides according to claim 1, each comprising a different sequence selected from SEQ ID NOs: 9, 1 to 8, 10 to 34 or a variant thereof; or
(b) two or more peptides that each are capable of binding to a different T cell receptor as defined in claim 19, wherein each different T cell receptor is capable of binding to a different sequence selected from SEQ ID NOs: 1 to 34 or a variant thereof.

32. A vaccine composition according to claim 30, which comprises:
(a) two or more polynucleotides each encoding a peptide according to claim 1, wherein each peptide comprises a different sequence selected from SEQ ID NOs: 1 to 34 or a variant thereof; or
(b) two or more polynucleotides each encoding a peptide that is capable of binding to a T cell receptor as defined in claim 18, wherein each peptide is capable of binding to a different T cell receptor as defined in claim 18 and each different T cell receptor is capable of binding to a different sequence selected from SEQ ID NOs: 1 to 34 or a variant thereof.

33. A method of preventing or treating a coronavirus infection, comprising administering a vaccine composition according to any one of claims 20 to 32 to an individual infected with, or at risk of being infected with, a coronavirus.

34. A vaccine composition according to any one of claims 20 to 32 for use in a method of preventing or treating a coronavirus infection in an individual.

35. A use according to any one of claims 8 to 18, a T cell receptor as defined in claim 19, a vaccine composition according to any one of claims 20 to 32, a method of preventing or treating a coronavirus infection according to claim 33, or a vaccine composition for use according to claim 34, wherein the coronavirus is SARS coronavirus or SARS coronavirus 2.


SEQUENCE LISTING (44 sequences)

SEQ ID NO: 1 - protein, 10 aa, Artificial Sequence
  synthetic construct
LLNKHIDAYK

SEQ ID NO: 2 - protein, 10 aa, Artificial Sequence
  synthetic construct
APSASAFFGM

SEQ ID NO: 3 - protein, 9 aa, Artificial Sequence
  synthetic construct
AQFAPSASA

SEQ ID NO: 4 - protein, 10 aa, Artificial Sequence
  synthetic construct
AQFAPSASAF

SEQ ID NO: 5 - protein, 9 aa, Artificial Sequence
  synthetic construct
ASAFFGMSR

SEQ ID NO: 6 - protein, 9 aa, Artificial Sequence
  synthetic construct
EVTPSGTWL

SEQ ID NO: 7 - protein, 9 aa, Artificial Sequence
  synthetic construct
FAPSASAFF

SEQ ID NO: 8 - protein, 9 aa, Artificial Sequence
  synthetic construct
GMSRIGMEV

SEQ ID NO: 9 - protein, 9 aa, Artificial Sequence
  synthetic construct
HLRIAGHHL

SEQ ID NO: 10 - protein, 9 aa, Artificial Sequence
  synthetic construct
KHWPQIAQF

SEQ ID NO: 11 - protein, 9 aa, Artificial Sequence
  synthetic construct
KTFPPTEPK

SEQ ID NO: 12 - protein, 10 aa, Artificial Sequence
  synthetic construct
KTFPPTEPKK

SEQ ID NO: 13 - protein, 9 aa, Artificial Sequence
  synthetic construct
LITGRLQSL

SEQ ID NO: 14 - protein, 9 aa, Artificial Sequence
  synthetic construct
LLLDRLNQL

SEQ ID NO: 15 - protein, 9 aa, Artificial Sequence
  synthetic construct
LLNKHIDAY

SEQ ID NO: 16 - protein, 9 aa, Artificial Sequence
  synthetic construct
MEVTPSGTW

SEQ ID NO: 17 - protein, 10 aa, Artificial Sequence
  synthetic construct
MEVTPSGTWL

SEQ ID NO: 18 - protein, 9 aa, Artificial Sequence
  synthetic construct
QFAPSASAF

SEQ ID NO: 19 - protein, 10 aa, Artificial Sequence
  synthetic construct
QFAPSASAFF

SEQ ID NO: 20 - protein, 9 aa, Artificial Sequence
  synthetic construct
QIAQFAPSA

SEQ ID NO: 21 - protein, 9 aa, Artificial Sequence
  synthetic construct
RLNEVAKNL

SEQ ID NO: 22 - protein, 9 aa, Artificial Sequence
  synthetic construct
SAFFGMSRI

SEQ ID NO: 23 - protein, 10 aa, Artificial Sequence
  synthetic construct
SASAFFGMSR

SEQ ID NO: 24 - protein, 9 aa, Artificial Sequence
  synthetic construct
SMWALIISV

SEQ ID NO: 25 - protein, 10 aa, Artificial Sequence
  synthetic construct
TKAYNVTQAF

SEQ ID NO: 26 - protein, 10 aa, Artificial Sequence
  synthetic construct
TLACFVLAAV

SEQ ID NO: 27 - protein, 9 aa, Artificial Sequence
  synthetic construct
TPSGTWLTY

SEQ ID NO: 28 - protein, 10 aa, Artificial Sequence
  synthetic construct
VTPSGTWLTY

SEQ ID NO: 29 - protein, 9 aa, Artificial Sequence
  synthetic construct
GETALALLL

SEQ ID NO: 30 - protein, 9 aa, Artificial Sequence
  synthetic construct
QFKDNVILL

SEQ ID NO: 31 - protein, 9 aa, Artificial Sequence
  synthetic construct
SMWALVISV

SEQ ID NO: 32 - protein, 10 aa, Artificial Sequence
  synthetic construct
TKQYNVTQAF

SEQ ID NO: 33 - protein, 9 aa, Artificial Sequence
  synthetic construct
GDAALALLL

SEQ ID NO: 34 - protein, 9 aa, Artificial Sequence
  synthetic construct
HLRMAGHSL

SEQ ID NO: 35 - protein, 12 aa, Artificial Sequence
  synthetic construct
AAYRLNEVAKNL

SEQ ID NO: 36 - protein, 13 aa, Artificial Sequence
  synthetic construct
AAYLLNKHIDAYK

SEQ ID NO: 37 - protein, 13 aa, Artificial Sequence
  synthetic construct
AAYQFAPSASAFF

SEQ ID NO: 38 - protein, 13 aa, Artificial Sequence
  synthetic construct
AAYVTPSGTWLTY

SEQ ID NO: 39 - protein, 13 aa, Artificial Sequence
  synthetic construct
AAYAPSASAFFGM

SEQ ID NO: 40 - protein, 12 aa, Artificial Sequence
  synthetic construct
AAYTPSGTWLTY

SEQ ID NO: 41 - protein, 12 aa, Artificial Sequence
  synthetic construct
AAYMEVTPSGTW

SEQ ID NO: 42 - protein, 12 aa, Artificial Sequence
  synthetic construct
AAYLLLDRLNQL

SEQ ID NO: 43 - protein, 4 aa, Artificial Sequence
  synthetic construct
LAAY

SEQ ID NO: 44 - protein, 5 aa, Artificial Sequence
  synthetic construct
FLAAY